SEQUENCE LISTING (8 sequences)

SEQ ID NO: 1 - protein, 443 aa, Synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 440 aa, Synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 214 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 444 aa, Synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 214 aa, Synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0325631 A1
(43) Pub. Date: Oct. 22, 2026

(54) Methods of Treating Cancer with Anti-PD-1 Agents

(71) Applicant: Tesaro, Inc., Wilmington, DE (US)

(72) Inventors: Srimoyee GHOSH, Waltham, MA (US); Rumen KOSTADINOV, Collegeville, PA (US)

(73) Assignee: Tesaro, Inc., Wilmington, DE (US)

(21) Appl. No.: 19/636,984

(22) Filed: Apr. 2, 2026

Related U.S. Application Data
(63) Continuation of application No. PCT/US2024/051899, filed on Oct. 18, 2024.
(60) Provisional application No. 63/591,485, filed on Oct. 19, 2023.
(60) Provisional application No. 63/637,555, filed on Apr. 23, 2024.

(51) Int. Cl. A61K 39/395; A61K 31/282; A61K 31/337; A61P 35/00

(52) U.S. Cl. CPC A61K 39/3955; A61K 31/282; A61K 31/337; A61P 35/00

(57) ABSTRACT

The disclosure provides a method of treating cancer in a patient by the administration of certain PD-1 antagonists. The disclosure also provides treatment regimens for patients previously treated with one or more chemotherapeutic agents or with radiation therapy.

### CROSS-REFERENCE TO RELATED APPLICATIONS

This is a continuation of International Patent Application No. PCT/US2024/051899, filed Oct. 18, 2024, which claims the benefit under 35 USC 119 (e) of U.S. Provisional Patent Application No. 63/637,555, filed Apr. 23, 2024, and U.S. Provisional Patent Application No. 63/591,485, filed Oct. 19, 2023. These applications are incorporated by reference herein.

### REFERENCE TO SEQUENCE LISTING

The instant application contains a sequence listing which has been submitted electronically in XML format and is hereby incorporated by reference in its entirety. Said XML copy, created on Mar. 19, 2026, is named “TES00066W001 XML Seq List” and is 10,571 bytes in size.

### FIELD

The present disclosure generally relates to methods of treating TP53 mutated cancer in a patient by the administration of certain PD-1 agents, such as antagonists. Treatment regimens for patients previously treated with one or more chemotherapeutic agents or with radiation therapy are also disclosed.

### BACKGROUND

Cancer is a serious public health problem, with about 609,360 people in the United States of America expected to die of cancer in 2022 alone according to the American Cancer Society, Cancer Facts & Figures 2022 (www.cancer.org/content/dam/cancer-org/research/cancer-facts-and-statistics/annual-cancer-facts-and-figures/2022/2022-cancer-facts-and-figures.pdf). Accordingly, there continues to be a need for effective therapies to treat cancer patients. TP53 is a well-known tumor suppressor gene, first cloned in 1979 with the genome keeping functions slowly elucidated in the subsequent decades leading up to 2000. Despite decades of research, there are still no TP53-directed cancer therapies.

### SUMMARY

In one aspect, the disclosure provides a method or treating TP53 mutant cancer in a human subject, the method comprising: administering a therapeutically effective amount of a combination of an anti-PD-1 agent, a platinum-based chemotherapy, and paclitaxel to the human patient, such as wherein the platinum-based chemotherapy is cisplatin or carboplatin. In one aspect, the disclosure provides a method of treating cancer in a human patient, the method comprising: administering a therapeutically effective amount of a combination comprising 500 mg to 1000 mg dostarlimab or a biosimilar thereof, a platinum-based chemotherapy, and paclitaxel to the human patient, such as wherein the platinum-based chemotherapy is cisplatin or carboplatin. In an embodiment, the platinum-based chemotherapy is carboplatin and wherein the cancer is a TP53-mutated cancer.

In an embodiment, the administering comprises a first dose of 500 mg dostarlimab or a biosimilar thereof once every 3 weeks (Q3W) for 4 to 6 cycles, followed by a second dose of 1000 mg dostarlimab or a biosimilar thereof once every 6 weeks (Q6W). In an embodiment, the second dose of dostarlimab or a biosimilar thereof is Q6W for up to 3 years. In an embodiment, the first and/or second doses of dostarlimab or a biosimilar thereof is by intravenous infusion, such as by intravenous infusion over 30 minutes. In an embodiment, the administering comprises a dose of carboplatin at an area under the concentration time curve of 5 milligram/milliliters/minute (mg/mL/min) once every 3 weeks (Q3W) for 4 to 6 cycles. In an embodiment, the administering comprises a dose of 175 milligram per meter square (mg/m²) paclitaxel once every 3 weeks (Q3W) for 4 to 6 cycles and wherein the cancer is a TP53-mutated cancer.

In an embodiment, the administering comprises: a first dose of 500 mg dostarlimab or a biosimilar thereof once every 3 weeks (Q3W) for 6 cycles, a dose of carboplatin at an area under the concentration time curve of 5 milligram/milliliters/minute (mg/mL/min) once every 3 weeks (Q3W) for 6 cycles, and a dose of 175 milligram per meter square (mg/m²) paclitaxel once every 3 weeks (Q3W) for 6 cycles, followed by a second dose of 1000 mg dostarlimab or a biosimilar thereof once every 6 weeks (Q6W). In an embodiment, the first dose of dostarlimab or a biosimilar thereof, the dose of carboplatin, and the dose of paclitaxel are all administered on day 1 of a 21-day cycle. In an embodiment, the second dose of dostarlimab or a biosimilar thereof is administered on day 1 of a 42-day cycle.

In an embodiment, the patient has not previously received treatment for the cancer. In an embodiment, the patient has not previously received an immunotherapy treatment for the cancer.

In an embodiment, the cancer is gynecologic cancer. In an embodiment, the gynecologic cancer is selected from endometrial cancer, ovarian cancer, fallopian tube cancer, cervical cancer, vaginal cancer, vulvar cancer, uterine cancer, uterine papillary serous carcinoma, primary peritoneal cancer, adnexal tumor, gestational trophoblastic tumor, or breast cancer. In an embodiment, the gynecologic cancer is endometrial cancer. In an embodiment, the cancer is tumor protein 53 (TP53) mutated cancer. In an embodiment, the cancer is tumor protein 53 (TP53) mutated endometrial cancer. In an embodiment, the cancer is mismatch repair deficient (dMMR) endometrial cancer. In an embodiment, the cancer is microsatellite instability-high (MSI-H) endometrial cancer. In an embodiment, the cancer is recurrent or advanced endometrial cancer. In an embodiment, the cancer is primary advanced endometrial cancer. In an embodiment, the cancer is stage III or stage IV endometrial cancer.

In an embodiment, the administering results in improved progression free survival (PFS) relative to administering the platinum-based chemotherapy and paclitaxel in the absence of dostarlimab or a biosimilar thereof. In an embodiment, the administering results in improved overall survival (OS) relative to administering the platinum-based chemotherapy and paclitaxel in the absence of dostarlimab or a biosimilar thereof. In an embodiment, the administering results in improved duration of response (DoR) relative to administering the platinum-based chemotherapy and paclitaxel in the absence of dostarlimab or a biosimilar thereof.

In one aspect, the disclosure provides a method of treating cancer in a human patient previously treated with one or more chemotherapeutic agents and/or with radiation therapy, the method comprising administering a therapeutically effective amount of dostarlimab or a biosimilar thereof to the human patient.

In an embodiment, the chemotherapeutic agent is a platinum-based chemotherapy. In an embodiment, the platinum-based chemotherapy is cisplatin or carboplatin.

In an embodiment, the patient discontinued administration of the radiation therapy prior to beginning administration of dostarlimab or a biosimilar thereof. In an embodiment, the patient discontinued administration of the one or more chemotherapeutic agents prior to beginning administration of dostarlimab or a biosimilar thereof.

In an embodiment, a platinum-based chemotherapy and paclitaxel are co-administered to the patient with dostarlimab or a biosimilar thereof, such as wherein the platinum-based chemotherapy is cisplatin or carboplatin. In an embodiment, the platinum-based chemotherapy is carboplatin.

In an embodiment, the administering comprises a first dose of 500 mg dostarlimab or a biosimilar thereof once every 3 weeks (Q3W) for 4 to 6 cycles, followed by a second dose of 1000 mg dostarlimab or a biosimilar thereof once every 6 weeks (Q6W). In an embodiment, the second dose of dostarlimab or a biosimilar thereof is Q6W for up to 3 years. In an embodiment, the first and/or second doses of dostarlimab or a biosimilar thereof is by intravenous infusion, such as by intravenous infusion over 30 minutes. In an embodiment, the administering comprises a dose of carboplatin at an area under the concentration time curve of 5 milligram/milliliters/minute (mg/mL/min) once every 3 weeks (Q3W) for 4 to 6 cycles. In an embodiment, the administering comprises a dose of 175 milligram per meter square (mg/m²) paclitaxel once every 3 weeks (Q3W) for 4 to 6 cycles.

In an embodiment, the administering comprises: a first dose of 500 mg dostarlimab or a biosimilar thereof once every 3 weeks (Q3W) for 6 cycles, a dose of carboplatin at an area under the concentration time curve of 5 milligram/milliliters/minute (mg/mL/min) once every 3 weeks (Q3W) for 6 cycles, and a dose of 175 milligram per meter square (mg/m²) paclitaxel once every 3 weeks (Q3W) for 6 cycles, followed by a second dose of 1000 mg dostarlimab or a biosimilar thereof once every 6 weeks (Q6W). In an embodiment, the first dose of dostarlimab or a biosimilar thereof, the dose of carboplatin, and the dose of paclitaxel are all administered on day 1 of a 21-day cycle. In an embodiment, the second dose of dostarlimab or a biosimilar thereof is administered on day 1 of a 42-day cycle.

In an embodiment, the cancer is gynecologic cancer. In an embodiment, the gynecologic cancer is selected from endometrial cancer, ovarian cancer, fallopian tube cancer, cervical cancer, vaginal cancer, vulvar cancer, uterine cancer, uterine papillary serous carcinoma, primary peritoneal cancer, adnexal tumor, gestational trophoblastic tumor, or breast cancer. In an embodiment, the gynecologic cancer is endometrial cancer. In an embodiment, the cancer is mismatch repair deficient (dMMR) endometrial cancer. In an embodiment, the cancer is microsatellite instability-high (MSI-H) endometrial cancer. In an embodiment, the cancer is recurrent or advanced endometrial cancer. In an embodiment, the cancer is primary advanced endometrial cancer. In an embodiment, the cancer is stage III or stage IV endometrial cancer.

In an embodiment, the administering results in improved progression free survival (PFS) relative to administering the platinum-based chemotherapy and paclitaxel in the absence of dostarlimab or a biosimilar thereof. In an embodiment, the administering results in improved overall survival (OS) relative to administering the platinum-based chemotherapy and paclitaxel in the absence of dostarlimab or a biosimilar thereof. In an embodiment, the administering results in improved duration of response (DoR) relative to administering the platinum-based chemotherapy and paclitaxel in the absence of dostarlimab or a biosimilar thereof.

In one aspect, the disclosure provides a method of treating cancer in a human patient, the method comprising: (a) selecting a human patient previously treated with one or more chemotherapeutic agents and/or with radiation therapy; and (b) administering a therapeutically effective amount of dostarlimab or a biosimilar thereof to the patient.

In an embodiment, the chemotherapeutic agent is a platinum-based chemotherapy. In an embodiment, the platinum-based chemotherapy is cisplatin or carboplatin.

In an embodiment, the patient discontinued administration of the radiation therapy prior to beginning administration of dostarlimab or a biosimilar thereof. In an embodiment, the patient discontinued administration of the one or more chemotherapeutic agents prior to beginning administration of dostarlimab or a biosimilar thereof.

In an embodiment, a platinum-based chemotherapy and paclitaxel are co-administered to the patient with dostarlimab or a biosimilar thereof, such as wherein the platinum-based chemotherapy is cisplatin or carboplatin. In an embodiment, the platinum-based chemotherapy is carboplatin.

In an embodiment, the administering comprises a first dose of 500 mg dostarlimab or a biosimilar thereof once every 3 weeks (Q3W) for 4 to 6 cycles, followed by a second dose of 1000 mg dostarlimab or a biosimilar thereof once every 6 weeks (Q6W). In an embodiment, the second dose of dostarlimab or a biosimilar thereof is Q6W for up to 3 years. In an embodiment, administration of the first and/or second doses of dostarlimab or a biosimilar thereof is by intravenous infusion, such as by intravenous infusion over 30 minutes. In an embodiment, the administering comprises a dose of carboplatin at an area under the concentration time curve of 5 milligram/milliliters/minute (mg/mL/min) once every 3 weeks (Q3W) for 4 to 6 cycles. In an embodiment, the administering comprises a dose of 175 milligram per meter square (mg/m²) paclitaxel once every 3 weeks (Q3W) for 4 to 6 cycles.

In an embodiment, the administering comprises: a first dose of 500 mg dostarlimab or a biosimilar thereof once every 3 weeks (Q3W) for 6 cycles, a dose of carboplatin at an area under the concentration time curve of 5 milligram/milliliters/minute (mg/mL/min) once every 3 weeks (Q3W) for 6 cycles, and a dose of 175 milligram per meter square (mg/m²) paclitaxel once every 3 weeks (Q3W) for 6 cycles, followed by a second dose of 1000 mg dostarlimab or a biosimilar thereof once every 6 weeks (Q6W). In an embodiment, the first dose of dostarlimab or a biosimilar thereof, the dose of carboplatin, and the dose of paclitaxel are all administered on day 1 of a 21-day cycle. In an embodiment, the second dose of dostarlimab or a biosimilar thereof is administered on day 1 of a 42-day cycle.

In an embodiment, the cancer is gynecologic cancer. In an embodiment, the gynecologic cancer is selected from endometrial cancer, ovarian cancer, fallopian tube cancer, cervical cancer, vaginal cancer, vulvar cancer, uterine cancer, uterine papillary serous carcinoma, primary peritoneal cancer, adnexal tumor, gestational trophoblastic tumor, or breast cancer. In an embodiment, the gynecologic cancer is endometrial cancer. In an embodiment, the cancer is mismatch repair deficient (dMMR) endometrial cancer. In an embodiment, the cancer is microsatellite instability-high (MSI-H) endometrial cancer. In an embodiment, the cancer is recurrent or advanced endometrial cancer. In an embodiment, the cancer primary advanced endometrial cancer. In an embodiment, the endometrial cancer is stage III or stage IV endometrial cancer.

In an embodiment, the administering results in improved progression free survival (PFS) relative to administering the platinum-based chemotherapy and paclitaxel in the absence of dostarlimab or a biosimilar thereof. In an embodiment, the administering results in improved overall survival (OS) relative to administering the platinum-based chemotherapy and paclitaxel in the absence of dostarlimab or a biosimilar thereof. In an embodiment, the administering results in improved duration of response (DoR) relative to administering the platinum-based chemotherapy and paclitaxel in the absence of dostarlimab or a biosimilar thereof.

In one aspect, the disclosure provides a method of treating gynecologic cancer in a human patient who has not received prior immunotherapy treatment, the method comprising administering a therapeutically effective amount of a combination comprising 500 mg to 1000 mg dostarlimab or a biosimilar thereof, a platinum-based chemotherapy, and paclitaxel to the patient. In an embodiment, the gynecologic cancer is a microsatellite instability-high (MSI-H) or mismatch repair deficient (dMMR) gynecologic cancer.

It is understood that any and all embodiments of the present disclosure may be taken in combination with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### BRIEF DESCRIPTION OF THE FIGURES

FIGS. 1A-1C are graphs depicting progression free survival (PFS) per investigator assessment of dostarlimab plus chemotherapy compared to placebo plus chemotherapy for dMMR/MSI-H patients (FIG. 1A), all comers (dMMR/MSI-H patients and MMRp/MSS patients) (FIG. 1B), and MMRp/MSS patients (FIG. 1C).

FIG. 2 shows progression free survival (PFS) per blinded independent central review (BICR) of dostarlimab plus chemotherapy compared to placebo plus chemotherapy.

FIGS. 3A-3C are graphs depicting overall survival (OS) of dostarlimab plus chemotherapy compared to placebo plus chemotherapy for all comers (dMMR/MSI-H patients and MMRp/MSS patients) (FIG. 3A), dMMR/MSI-H patients (FIG. 3B), and MMRp/MSS patients (FIG. 3C).

FIGS. 4A-4C are graphs depicting duration of response (DoR) per investigator assessment according to RECIST 1.1 of dostarlimab plus chemotherapy compared to placebo plus chemotherapy for dMMR/MSI-H patients (FIG. 4A), all comers (dMMR/MSI-H patients and MMRp/MSS patients) (FIG. 4B), and MMRp/MSS patients (FIG. 4C).

FIG. 5 shows the diagnostic scheme for the endometrial (EC) subclasses. Footnote a: Pathogenic POLE variants include Pro286Arg, Val411Leu, Ser297Phe, Ala456Pro and Ser459Phe; Footnote b: dMMR or MS-H; Footnote c: TP53 mutations assessed by WES; Footnote *: NSMP, no specific molecular profile.

FIG. 6 shows that Dostarlimab treatment shows significant PFS benefit in dMMR/MSI-H molecular subgroup (Stratified Cox model, p=0)

FIG. 7A shows Kaplan-Meier Progression Free Survival (PFS) curves for all molecular subgroups. Censoring is shown as embedded ‘+’ marks on the various curves. Dostarlimab+Carboplatin-Paclitaxel groups (IO) are solid lines; Placebo+Carboplatin-Paclitaxel groups (SOC) are dashed lines. IO patient subgroup curves: POLE-mut patients, red solid line; dMMR/MSI-H, green solid line; NSMP (no specific molecular profile) blue solid line; TP53-mut pink solid line. SOC patient subgroup curves: POLE-mut, brown dashed lines; dMMR/MSI-H, dark green dashed lines; NSMP, dark red dashed lines; TP53-mut, dark red dashed lines.

FIG. 7B shows Kaplan-Meier Overall Survival (OS) curves for all molecular subgroups. Censoring is shown as embedded ‘+’ marks on the various curves. Dostarlimab+Carboplatin-Paclitaxel groups (IO) are solid lines; Placebo+Carboplatin-Paclitaxel groups (SOC) are dashed lines. IO patient subgroup curves: POLE-mut patients, red solid line; dMMR/MSI-H, green solid line; NSMP (no specific molecular profile) blue solid line; TP53-mut pink solid line. SOC patient subgroup curves: POLE-mut, brown dashed lines; dMMR/MSI-H, dark green dashed lines; NSMP, dark red dashed lines; TP53-mut, dark red dashed lines.

FIG. 8 Kaplan-Meier Progression Free Survival (PFS) curves for TP53-mut molecular subgroup showing a significant dostarlimab treatment benefit (Stratified Cox model, p=0.04; Hazard Ratio 0.55 (95% CI, 0.3-0.99)). Dostarlimab+Carboplatin-Paclitaxel K-M curve gold solid line; Placebo+Carboplatin-Paclitaxel K-M curve blue solid line. Patient censoring events shown as ‘+’ marks on the curves.

FIG. 9 Kaplan-Meier Overall Survival (OS) curves for TP53-mut molecular subgroup showing a significant dostarlimab treatment benefit (Stratified Cox model, p=0.01; Hazard Ratio 0.41 (95% CI, 0.2-0.82)). Dostarlimab+Carboplatin-Paclitaxel K-M curve gold solid line; Placebo+Carboplatin-Paclitaxel K-M curve blue solid line. Patient censoring events shown as ‘+’ marks on the curves.

FIG. 10 Kaplan-Meier Progression Free Survival (PFS) curves for the NSMP subgroup. Dostarlimab+Carboplatin-Paclitaxel K-M curve gold solid line; Placebo+Carboplatin-Paclitaxel K-M curve blue solid line. Patient censoring events shown as ‘+’ marks on the curves. There is a trend for some benefit from Dostarlimab+Carboplatin-Paclitaxel over Placebo+Carboplatin-Paclitaxel: Stratified Cox model, p=0.12 and Hazard Ratio 0.77 (95% CI, 0.55-1.07).

FIG. 11 Kaplan-Meier Overall Survival (OS) curves for the NSMP subgroup. Dostarlimab+Carboplatin-Paclitaxel K-M curve gold solid line; Placebo+Carboplatin-Paclitaxel K-M curve blue solid line. Patient censoring events shown as ‘+’ marks on the curves. No measured benefit from Dostarlimab+Carboplatin-Paclitaxel over Placebo+Carboplatin-Paclitaxel: Stratified Cox model, p=0.54 and Hazard Ratio 0.87 (95% CI, 0.56-1.36).

FIG. 12 Kaplan-Meier PFS curves for Dostarlimab treatment shows significant PFS benefit in dMMR/MSI-H molecular subgroup: Stratified Cox model, p=0; Hazard Ratio 0.31 (95% CI 0.17-0.56). Dostarlimab+Carboplatin-Paclitaxel K-M curve gold solid line; Placebo+Carboplatin-Paclitaxel K-M curve blue solid line. Patient censoring events shown as ‘+’ marks on the curves.

FIG. 13 Kaplan-Meier OS curves for Dostarlimab treatment shows significant OS benefit in dMMR/MSI-H subgroup: Stratified Cox model, p=0.04; Hazard Ratio 0.4 (95% CI 0.17-0.95).

### DETAILED DESCRIPTION

### Definitions

As used herein and in the claims, the term “comprising” encompasses “including” or “consisting,” e.g., a composition “comprising” X may consist exclusively of X or may include something additional, e.g., X+Y.

Unless defined otherwise, all technical and scientific terms used herein have the same meaning as commonly understood by one of ordinary skill in the art to which this disclosure pertains. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods and uses disclosed herein.

Concentrations described herein are determined at ambient temperature and pressure. This may be, for example, the temperature and pressure at room temperature or within a particular portion of a process stream. In general, concentrations are determined at a standard state of 25° C. and 1 bar of pressure.

As used herein, the term “about” means within +10% of the stated value.

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long): an amino-terminal variable (VH) domain (located at the tips of the Y structure), followed by three constant domains: CH1, CH2, and the carboxy-terminal CH3 (located at the base of the Y's stem). A short region, known as the “switch,” connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains: an amino-terminal variable (VL) domain, followed by a carboxy-terminal constant (CL) domain, separated from one another by another “switch.” Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3—, 4—, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered glycosylation. For purposes of the present disclosure, in certain embodiments, any polypeptide or complex of polypeptides that includes sufficient immunoglobulin domain sequences as found in natural antibodies can be referred to and/or used as an “antibody,” whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, fully human, and the like, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi-specific antibodies; antibody fragments such as Fab fragments, Fab′ fragments, F(ab′)2 fragments, Fd′ fragments, Fd fragments, and isolated CDRs or sets thereof; single chain Fvs; polypeptide-Fc fusions; and single domain antibodies. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, and the like), or other pendant group (e.g., poly-ethylene glycol, and the like)).

As used herein, the term “progression free survival” means the time period for which a subject having a disease (e.g., cancer) survives, without a significant worsening of the disease state. Progression free survival may be assessed as a period of time in which there is no progression of tumor growth and/or wherein the disease status of a patient is not determined to be a progressive disease. In some embodiments, progression free survival of a subject having cancer is assessed by evaluating tumor (lesion) size, tumor (lesion) number, and/or metastasis.

The term “progression” of tumor growth or a “progressive disease” (“PD”) as used herein in reference to cancer status indicates an increase in the sum of the diameters of the target lesions (tumors). In some embodiments, progression of tumor growth refers to at least a 20% increase in the sum of diameters of target lesions, taking as reference the smallest sum on study (this includes the baseline sum if that is the smallest on study). In some embodiments, in addition to a relative increase of 20%, the sum of diameters of target lesions must also demonstrate an absolute increase of at least 5 mm. An appearance of one or more new lesions may also be factored into the determination of progression of tumor growth. Progression for the purposes of determining progression free survival may also be determined if at least one of the following criteria is met: 1) tumor assessment by CT/MRI unequivocally shows progressive disease according to RECIST 1.1 or irRECIST criteria; or 2) additional diagnostic tests (e.g., histology/cytology, ultrasound techniques, endoscopy, positron emission tomography) identify new lesions or determine existing lesions qualify for unequivocal progressive disease AND CA-125-progression according to Gynecologic Cancer Intergroup (GCIG)-criteria (see Rustin et al., Int J Gynecol Cancer 2011; 21:419-423 which is incorporated herein in its entirety); 3) definitive clinical signs and symptoms of PD unrelated to non-malignant or iatrogenic causes ([i] intractable cancer-related pain; [ii] malignant bowel obstruction/worsening dysfunction; or [iii] unequivocal symptomatic worsening of ascites or pleural effusion) AND CA-125-progression according to GCIG-criteria.

As used herein, the term “complete response” or “CR” is used to mean the disappearance of all or substantially all target lesions. Any pathological lymph nodes must be <10 mm in the short axis. In some embodiments, CR refers to an about 80%, 85%, 90%, 91%, 92%, 93%, 94%, 95%, 96%, 97%, 98%, 99% or 100% decrease in the sum of the diameters of the target lesions (i.e., loss of lesions), taking as reference the baseline sum diameters. In some embodiments, CR indicates that less than about 10%, 9%, 8%, 7%, 6%, 5%, 4%, 3%, 2%, 1% or less of the total lesion diameter remains after treatment. Exemplary methods for evaluating complete response are identified by RECIST guidelines. See, e.g., E. A. Eisenhauer, et al., “New response evaluation criteria in solid tumors: Revised RECIST guideline (version 1.1.),” Eur. J. of Cancer, 45:228-247 (2009).

As used herein, the term “partial response” or “PR” refers to a decrease in tumor progression in a subject as indicated by a decrease in the sum of the diameters of the target lesions, taking as reference the baseline sum diameters. In some embodiments, PR refers to at least a 30% decrease in the sum of diameters of target lesions, taking as reference the baseline sum diameters. Exemplary methods for evaluating partial response are identified by RECIST guidelines. See e.g., E. A. Eisenhauer, et al., “New response evaluation criteria in solid tumors: Revised RECIST guideline (version 1.1.),” Eur. J. of Cancer, 45:228-247 (2009).

As used herein, “stabilization” of tumor growth or a “stable disease” (“SD”) refers to neither sufficient shrinkage to qualify for PR nor sufficient increase to qualify for PD. In some embodiments, stabilization refers to a less than 30%, 25%, 20%, 15%, 10% or 5% change (increase or decrease) in the sum of the diameters of the target lesions, taking as reference the baseline sum diameters. Exemplary methods for evaluating stabilization of tumor growth or a stable disease are identified by RECIST guidelines. See e.g., E. A. Eisenhauer, et al., “New response evaluation criteria in solid tumors: Revised RECIST guideline (version 1.1.),” Eur. J. of Cancer, 45:228-247 (2009).

As used herein, the term “patient” means a human patient.

### Methods of Treating Cancer

Described herein are methods of treating cancer in a patient that benefits from administration of PD-1 agents, such as antagonists. Also described are uses of PD-1 antagonists in the treatment of cancer in a patient.

In one aspect, the disclosure provides methods of treating cancer in a human patient, the method comprising: administering a therapeutically effective amount of a combination comprising a PD-1 antagonist (e.g., an anti-PD-1 antibody) and chemotherapy to the human patient, such as wherein the chemotherapy is a platinum-based chemotherapy and paclitaxel.

In one aspect, the disclosure provides methods of treating cancer in a human patient previously treated with one or more chemotherapeutic agents and/or with radiation therapy, the method comprising administering a therapeutically effective amount of a PD-1 antagonist (e.g., an anti-PD-1 antibody to the human patient.

In one aspect, the disclosure provides methods of treating cancer in a human patient, the method comprising: (a) selecting a human patient previously treated with one or more chemotherapeutic agents and/or with radiation therapy; and (b) administering a therapeutically effective amount of a PD-1 antagonist (e.g., an anti-PD-1 antibody) to the patient.

In one aspect, the disclosure provides methods of treating gynecologic cancer in a human patient who has not received prior immunotherapy treatment, the method comprising administering a therapeutically effective amount of a combination comprising a PD-1 antagonist (e.g., an anti-PD-1 antibody) and chemotherapy to the patient, such as wherein the chemotherapy is a platinum-based chemotherapy and paclitaxel.

The present disclosure provides methods of treating cancer in a patient. In embodiments, a cancer is associated with a high tumor mutation burden (TMB). In embodiments, a cancer is microsatellite stable (MSS). In embodiments a cancer is characterized by microsatellite instability. In embodiments, a cancer has a high microsatellite instability status (MSI-H). In embodiments, a cancer has a low microsatellite instability status (MSI-L). In embodiments, a cancer is associated with high TMB and MSI-H. In embodiments, a cancer is associated with high TMB and MSI-L or MSS. In embodiments, a cancer is associated with high TMB and MSI-L. In embodiments, a cancer is associated with high TMB and MSS. In embodiments, a cancer has a defective DNA mismatch repair system. In embodiments, a cancer has a defect in a DNA mismatch repair gene. In embodiments, a cancer is a hypermutated cancer. In embodiments, a cancer has homologous recombination repair deficiency/homologous repair deficiency (“HRD”). In embodiments, a cancer comprises a mutation in polymerase delta (POLD). In embodiments, a cancer comprises a mutation in polymerase epsilon (POLE).

In embodiments, a cancer is endometrial cancer (e.g., MSI-H or MSS/MSI-L endometrial cancer). In embodiments, a cancer is a MSI-H cancer comprising a mutation in POLE or POLD (e.g., a MSI-H non-endometrial cancer comprising a mutation in POLE or POLD). In embodiments, a cancer is breast cancer (e.g., triple negative breast cancer (TNBC)). In embodiments, a cancer is lung cancer (e.g., non-small cell lung cancer). In embodiments, a cancer is melanoma. In embodiments, a cancer is colorectal cancer. In embodiments, a cancer is squamous cell carcinoma of the anus, squamous cell carcinoma of the penis, squamous cell carcinoma of the cervix, squamous cell carcinoma of the vagina, or squamous cell carcinoma of the vulva.

In embodiments, a cancer is adenocarcinoma, endometrial cancer, breast cancer, ovarian cancer, cervical cancer, fallopian tube cancer, testicular cancer, primary peritoneal cancer, colon cancer, colorectal cancer, stomach cancer, small intestine cancer, squamous cell carcinoma of the anogenital region (e.g., squamous cell carcinoma of the anus, penis, cervix, vagina, or vulva), soft tissue sarcoma (e.g., leiomyosarcoma), melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor. In embodiments, a cancer is MSS or MSI-L, is characterized by microsatellite instability, is MSI-H, has high TMB, has high TMB and is MSS or MSI-L, has high TMB and is MSI-H, has a defective DNA mismatch repair system, has a defect in a DNA mismatch repair gene, is a hypermutated cancer, is an HRD cancer, comprises a mutation in polymerase delta (POLD) or comprises a mutation in polymerase epsilon (POLE).

In embodiments, a cancer has homologous recombination repair deficiency/homologous repair deficiency (“HRD”). In embodiments, a cancer is acute myeloid leukemia. In embodiments, a cancer is acute lymphoblastic leukemia. In embodiments, a cancer is non-Hodgkin's lymphoma. In embodiments, a cancer is Hodgkin's lymphoma. In embodiments, a cancer is neuroblastoma. In embodiments, a cancer is a CNS tumor. In embodiments, a cancer is diffuse intrinsic pontine glioma (DIPG). In embodiments, a cancer is Ewing's sarcoma. In embodiments, a cancer is embryonal rhabdomyosarcoma. In embodiments, a cancer is osteosarcoma. In embodiments, a cancer is Wilms tumor. In embodiments, a cancer is a soft tissue sarcoma (e.g., leiomyosarcoma).

In some embodiments, a patient has cancer, such as a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In some certain embodiments, a patient has an anal cancer, a fallopian tube cancer, an ovarian cancer, or a lung cancer. In some certain embodiments, a patient has a cancer of the anus. In some certain embodiments, a patient has a cancer of the fallopian tube(s). In some certain embodiments, a patient has an ovarian cancer. In some certain embodiments, a patient has a lung cancer.

In some embodiments, a patient has a cancer with microsatellite instability. In some embodiments, the microsatellite instability is considered high, wherein the instability is significantly higher than that observed in a control cell (e.g., MSI-H status). In some embodiments, the microsatellite instability is MSI-Low. In some embodiments, the microsatellite instability is microsatellite stable (e.g., MSS status). In some embodiments, a cancer with microsatellite instability is a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In some certain embodiments, a cancer with microsatellite instability is an anal cancer, a fallopian tube cancer, an ovarian cancer, or a lung cancer. In some certain embodiments, a patient has an endometrial cancer with microsatellite instability. In some embodiments, a patient has an endometrial cancer that is microsatellite stable (MSS).

In some embodiments, a patient has a cancer characterized by PD-1 and/or PD-L1 expression. In some embodiments, a cancer has high PD-1 and/or PD-L1 expression (e.g., by high PD-1 and/or high PD-L1 expression). In some embodiment, a cancer characterized by PD-1 and/or PD-L1 expression is a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In some certain embodiments, a cancer characterized by PD-1 and/or PD-L1 expression is an anal cancer, a fallopian tube cancer, an ovarian cancer, or a lung cancer.

In embodiments, a cancer is an advanced cancer. In embodiments, a cancer is a metastatic cancer. In embodiments, a cancer is a MSI-H cancer. In embodiments, a cancer is a MSS cancer. In embodiments, a cancer is a POLE-mutant cancer. In embodiments, a cancer is a POLD-mutant cancer. In embodiments, a cancer is a high TMB cancer. In embodiments, a cancer is associated with homologous recombination repair deficiency/homologous repair deficiency (“HRD”).

In embodiments, a cancer is a solid tumor. In embodiments, a solid tumor is advanced. In embodiments, a solid tumor is a metastatic solid tumor. In embodiments, a solid tumor is a MSI-H solid tumor. In embodiments, a solid tumor is a MSS solid tumor. In embodiments, a solid tumor is a POLE-mutant solid tumor. In embodiments, a solid tumor is a POLD-mutant solid tumor. In embodiments, a solid tumor is a high TMB solid tumor. In embodiments, a solid tumor is associated with homologous recombination repair deficiency/homologous repair deficiency (“HRD”).

In embodiments, a cancer is a non-endometrial cancer (e.g., a non-endometrial solid tumor). In embodiments, a non-endometrial cancer is an advanced cancer. In embodiments, a non-endometrial cancer is a metastatic cancer. In embodiments, a non-endometrial cancer is a MSI-H cancer. In embodiments, a non-endometrial cancer is a MSS cancer. In embodiments, a non-endometrial cancer is a POLE-mutant cancer. In embodiments, a non-endometrial cancer is a solid tumor (e.g., a MSS solid tumor, a MSI-H solid tumor, a POLD mutant solid tumor, or a POLE-mutant solid tumor). In embodiments, a non-endometrial cancer is a high TMB cancer. In embodiments, a non-endometrial cancer is associated with homologous recombination repair deficiency/homologous repair deficiency (“HRD”).

In embodiments, a cancer is endometrial cancer (e.g., a solid tumor). In embodiments, an endometrial cancer is an advanced cancer. In embodiments, an endometrial cancer is a metastatic cancer. In embodiments, an endometrial cancer is a MSI-H endometrial cancer. In embodiments, an endometrial cancer is a MSS endometrial cancer. In embodiments, an endometrial cancer is a POLE-mutant endometrial cancer. In embodiments, an endometrial cancer is a POLD-mutant endometrial cancer. In embodiments, an endometrial cancer is a high TMB endometrial cancer. In embodiments, an endometrial cancer is associated with homologous recombination repair deficiency/homologous repair deficiency (“HRD”).

In some embodiments, the cancer is a TP53 mutated cancer. In embodiments, a TP53 mutated cancer is endometrial cancer. In embodiments, a TP53 mutated cancer is endometrial carcinoma or an endometrial adenocarcinoma. In embodiments, a TP53 mutated cancer is an endometrioid adenocarcinoma. In embodiments, a TP53 mutated cancer is endometrial serous carcinoma. In embodiments, a TP53 mutated cancer is endometrial clear cell carcinoma. In embodiments, a TP53 mutated cancer is endometrial mucinous carcinoma. In embodiments, a TP53 mutated cancer is an endometrial mixed carcinoma of an endometrial undifferentiated carcinoma. In embodiments, a TP53 mutated cancer is a urothelial carcinoma or a transition cell carcinoma.

In embodiments, a cancer is a lung cancer (e.g., a solid tumor). In embodiments, a lung cancer is an advanced lung cancer. In embodiments, a lung cancer is a metastatic lung cancer. In embodiments, a lung cancer is squamous cell carcinoma of the lung. In embodiments, a lung cancer is small cell lung cancer (SCLC). In embodiments, a lung cancer is non-small cell lung cancer (NSCLC). In embodiments, a lung cancer is an ALK-translocated lung cancer (e.g., a lung cancer with a known ALK-translocation). In embodiments, a lung cancer is an EGFR-mutant lung cancer (e.g., a lung cancer with a known EGFR mutation). In embodiments, a lung cancer is a MSI-H lung cancer. In embodiments, a lung cancer is a MSS lung cancer. In embodiments, a lung cancer is a POLE-mutant lung cancer. In embodiments, a lung cancer is a POLD-mutant lung cancer. In embodiments, a lung cancer is a high TMB lung cancer. In embodiments, a lung cancer is associated with homologous recombination repair deficiency/homologous repair deficiency (“HRD”). In embodiments, a lung cancer is a TP53 mutated lung cancer.

In embodiments, a cancer is a colorectal (CRC) cancer (e.g., a solid tumor). In embodiments, a colorectal cancer is an advanced colorectal cancer. In embodiments, a colorectal cancer is a metastatic colorectal cancer. In embodiments, a colorectal cancer is a MSI-H colorectal cancer. In embodiments, a colorectal cancer is a MSS colorectal cancer. In embodiments, a colorectal cancer is a POLE-mutant colorectal cancer. In embodiments, a colorectal cancer is a POLD-mutant colorectal cancer. In embodiments, a colorectal cancer is a high TMB colorectal cancer. In embodiments, a colorectal cancer is associated with homologous recombination repair deficiency/homologous repair deficiency (“HRD”). In embodiments, a colorectal cancer is a TP53 mutated colorectal cancer.

In embodiments, a cancer is a melanoma. In embodiments, a melanoma is an advanced melanoma. In embodiments, a melanoma is a metastatic melanoma. In embodiments, a melanoma is a MSI-H melanoma. In embodiments, a melanoma is a MSS melanoma. In embodiments, a melanoma is a POLE-mutant melanoma. In embodiments, a melanoma is a POLD-mutant melanoma. In embodiments, a melanoma is a high TMB melanoma. In embodiments, a melanoma is associated with homologous recombination repair deficiency/homologous repair deficiency (“HRD”). In embodiments, a melanoma is a TP53 mutated melanoma.

In embodiments, a cancer is squamous cell carcinoma of the anogenital region (e.g., of the anus, penis, cervix, vagina, or vulva). In embodiments, a squamous cell carcinoma of the anogenital region (e.g., of the anus, penis, cervix, vagina, or vulva) is an advanced cancer. In embodiments, a squamous cell carcinoma of the anogenital region (e.g., of the anus, penis, cervix, vagina, or vulva) is a metastatic cancer. In embodiments, a squamous cell carcinoma of the anogenital region (e.g., of the anus, penis, cervix, vagina, or vulva) is MSI-H. In embodiments, a squamous cell carcinoma of the anogenital region (e.g., of the anus, penis, cervix, vagina, or vulva) is MSS. In embodiments, a lung cancer is a POLE-mutant cancer. In embodiments, a squamous cell carcinoma of the anogenital region (e.g., of the anus, penis, cervix, vagina, or vulva) is associated with homologous recombination repair deficiency/homologous repair deficiency (“HRD”). In embodiments, a squamous cell carcinoma of the anogenital region is a TP53 mutated squamous cell carcinoma of the anogenital region.

In embodiments, a cancer is an ovarian cancer. In embodiments, an ovarian cancer is an advanced ovarian cancer. In embodiments, an ovarian cancer is a metastatic ovarian cancer. In embodiments, an ovarian cancer is a MSI-H ovarian cancer. In embodiments, an ovarian cancer is a MSS ovarian cancer. In embodiments, an ovarian cancer is a POLE-mutant ovarian cancer. In embodiments, an ovarian cancer is a POLD-mutant ovarian cancer. In embodiments, an ovarian cancer is a high TMB ovarian cancer. In embodiments, an ovarian cancer is associated with homologous recombination repair deficiency/homologous repair deficiency (“HRD”). In embodiments, an ovarian cancer is a serous cell ovarian cancer. In embodiments, an ovarian cancer is a clear cell ovarian cancer. In embodiments, an ovarian cancer is a TP53 mutated ovarian cancer.

In embodiments, a cancer is a fallopian cancer. In embodiments, a fallopian cancer is an advanced fallopian cancer. In embodiments, a fallopian cancer is a metastatic fallopian cancer. In embodiments, a fallopian cancer is a MSI-H fallopian cancer. In embodiments, a fallopian cancer is a MSS fallopian cancer. In embodiments, a fallopian cancer is a POLE-mutant fallopian cancer. In embodiments, a fallopian cancer is a POLD-mutant fallopian cancer. In embodiments, a fallopian cancer is a high TMB fallopian cancer. In embodiments, a fallopian cancer is associated with homologous recombination repair deficiency/homologous repair deficiency (“HRD”). In embodiments, a fallopian cancer is a serous cell fallopian cancer. In embodiments, a fallopian cancer is a clear cell fallopian cancer. In embodiments, a fallopian cancer is a TP53 mutated fallopian cancer.

In embodiments, a cancer is a primary peritoneal cancer. In embodiments, a primary peritoneal cancer is an advanced primary peritoneal cancer. In embodiments, a primary peritoneal cancer is a metastatic primary peritoneal cancer. In embodiments, a primary peritoneal cancer is a MSI-H primary peritoneal cancer. In embodiments, a primary peritoneal cancer is a MSS primary peritoneal cancer. In embodiments, a primary peritoneal cancer is a POLE-mutant primary peritoneal cancer. In embodiments, a primary peritoneal cancer is a POLD-mutant primary peritoneal cancer. In embodiments, a primary peritoneal cancer is a high TMB primary peritoneal cancer. In embodiments, a primary peritoneal cancer is associated with homologous recombination repair deficiency/homologous repair deficiency (“HRD”). In embodiments, a primary peritoneal cancer is a serous cell primary peritoneal cancer. In embodiments, a primary peritoneal cancer is a clear cell primary peritoneal cancer. In embodiments, a primary peritoneal cancer is a TP53 mutated primary peritoneal cancer.

In embodiments, a cancer is acute lymphoblastic leukemia (“ALL”). In embodiments, acute lymphoblastic leukemia is advanced acute lymphoblastic leukemia. In embodiments, acute lymphoblastic leukemia is metastatic acute lymphoblastic leukemia. In embodiments, acute lymphoblastic leukemia is MSI-H acute lymphoblastic leukemia. In embodiments, acute lymphoblastic leukemia is MSS acute lymphoblastic leukemia. In embodiments, acute lymphoblastic leukemia is POLE-mutant acute lymphoblastic leukemia. In embodiments, acute lymphoblastic leukemia is POLD-mutant acute lymphoblastic leukemia. In embodiments, an acute lymphoblastic leukemia is associated with homologous recombination repair deficiency/homologous repair deficiency (“HRD”). In embodiments, an acute lymphoblastic leukemia is a TP53 mutated acute lymphoblastic leukemia.

In embodiments, a cancer is acute myeloid leukemia (“AML”). In embodiments, acute myeloid leukemia is advanced acute myeloid leukemia. In embodiments, acute myeloid leukemia is metastatic acute myeloid leukemia. In embodiments, acute myeloid leukemia is MSI-H acute myeloid leukemia. In embodiments, acute myeloid leukemia is MSS acute myeloid leukemia. In embodiments, acute myeloid leukemia is POLE-mutant acute myeloid leukemia. In embodiments, acute myeloid leukemia is POLD-mutant acute myeloid leukemia. In embodiments, an acute myeloid leukemia is associated with homologous recombination repair deficiency/homologous repair deficiency (“HRD”). In embodiments, acute myeloid leukemia is TP53 mutated acute myeloid leukemia.

In embodiments, a cancer is non-Hodgkin's lymphoma (NHL). In embodiments, non-Hodgkin's lymphoma is advanced non-Hodgkin's lymphoma. In embodiments, non-Hodgkin's lymphoma is metastatic non-Hodgkin's lymphoma. In embodiments, non-Hodgkin's lymphoma is MSI-H non-Hodgkin's lymphoma. In embodiments, non-Hodgkin's lymphoma is MSS non-Hodgkin's lymphoma. In embodiments, non-Hodgkin's lymphoma is POLE-mutant non-Hodgkin's lymphoma. In embodiments, non-Hodgkin's lymphoma is POLD-mutant non-Hodgkin's lymphoma. In embodiments, non-Hodgkin's lymphoma is associated with homologous recombination repair deficiency/homologous repair deficiency (“HRD”). In embodiments, non-Hodgkin's lymphoma is TP53 non-Hodgkin's lymphoma

In embodiments, a cancer is Hodgkin's lymphoma (HL). In embodiments, Hodgkin's lymphoma is advanced Hodgkin's lymphoma. In embodiments, Hodgkin's lymphoma is metastatic Hodgkin's lymphoma. In embodiments, Hodgkin's lymphoma is MSI-H Hodgkin's lymphoma. In embodiments, Hodgkin's lymphoma is MSS Hodgkin's lymphoma. In embodiments, Hodgkin's lymphoma is POLE-mutant Hodgkin's lymphoma. In embodiments, Hodgkin's lymphoma is POLD-mutant Hodgkin's lymphoma. In embodiments, Hodgkin's lymphoma is associated with homologous recombination repair deficiency/homologous repair deficiency (“HRD”). In embodiments, Hodgkin's lymphoma is TP53 Hodgkin's lymphoma.

In embodiments, a cancer is a neuroblastoma (NB). In embodiments, a neuroblastoma is an advanced neuroblastoma. In embodiments, a neuroblastoma is a metastatic neuroblastoma. In embodiments, neuroblastoma is a MSI-H neuroblastoma. In embodiments, a neuroblastoma is a MSS neuroblastoma. In embodiments, a neuroblastoma is a POLE-mutant neuroblastoma. In embodiments, a neuroblastoma is a POLD-mutant neuroblastoma. In embodiments, a neuroblastoma is a high TMB neuroblastoma. In embodiments, a neuroblastoma is associated with homologous recombination repair deficiency/homologous repair deficiency (“HRD”). In embodiments, neuroblastoma is a TP53 mutated neuroblastoma.

In embodiments, a cancer is a CNS tumor. In embodiments, a CNS tumor is advanced. In embodiments, a CNS tumor is a metastatic CNS tumor. In embodiments, a CNS tumor is a MSI-H CNS tumor. In embodiments, a CNS tumor is a MSS CNS tumor. In embodiments, a CNS tumor is a POLE-mutant CNS tumor. In embodiments, a CNS tumor is a POLD-mutant CNS tumor. In embodiments, a CNS tumor is a high TMB CNS tumor. In embodiments, a CNS tumor is associated with homologous recombination repair deficiency/homologous repair deficiency (“HRD”). In embodiments, a CNS tumor is a TP53 mutated CNS tumor.

In embodiments, a cancer is diffuse intrinsic pontine glioma (DIPG). In embodiments, a DIPG is an advanced DIPG. In embodiments, a DIPG is a metastatic DIPG. In embodiments, DIPG is a MSI-H DIPG. In embodiments, a DIPG is a MSS DIPG. In embodiments, a DIPG is a POLE-mutant DIPG. In embodiments, a DIPG is a POLD-mutant DIPG. In embodiments, a DIPG is a high TMB DIPG. In embodiments, a DIPG is associated with homologous recombination repair deficiency/homologous repair deficiency (“HRD”). In embodiments, a DIPG is a TP53 mutated DIPG.

In embodiments, a cancer is Ewing's sarcoma. In embodiments, Ewing's sarcoma is an advanced Ewing's sarcoma. In embodiments, Ewing's sarcoma is a metastatic Ewing's sarcoma. In embodiments, Ewing's sarcoma is a MSI-H Ewing's sarcoma. In embodiments, Ewing's sarcoma is a MSS Ewing's sarcoma. In embodiments, Ewing's sarcoma is a POLE-mutant Ewing's sarcoma. In embodiments, Ewing's sarcoma is a POLD-mutant Ewing's sarcoma. In embodiments, Ewing's sarcoma is a high TMB Ewing's sarcoma. In embodiments, Ewing's sarcoma is associated with homologous recombination repair deficiency/homologous repair deficiency (“HRD”). In embodiments, Ewing's sarcoma is a TP53 mutated Ewing's sarcoma.

In embodiments, a cancer is an embryonal rhabdomyosarcoma (ERS). In embodiments, an embryonal rhabdomyosarcoma is an advanced embryonal rhabdomyosarcoma. In embodiments, an embryonal rhabdomyosarcoma is a metastatic embryonal rhabdomyosarcoma. In embodiments, an embryonal rhabdomyosarcoma is a MSI-H embryonal rhabdomyosarcoma. In embodiments, an embryonal rhabdomyosarcoma is a MSS embryonal rhabdomyosarcoma. In embodiments, an embryonal rhabdomyosarcoma is a POLE-mutant embryonal rhabdomyosarcoma. In embodiments, an embryonal rhabdomyosarcoma is a POLD-mutant embryonal rhabdomyosarcoma. In embodiments, an embryonal rhabdomyosarcoma is a high TMB embryonal rhabdomyosarcoma. In embodiments, an embryonal rhabdomyosarcoma is associated with homologous recombination repair deficiency/homologous repair deficiency (“HRD”). In embodiments, an embryonal rhabdomyosarcoma is a TP53 mutated embryonal rhabdomyosarcoma.

In embodiments, a cancer is an osteosarcoma (OS). In embodiments, an osteosarcoma is an advanced osteosarcoma. In embodiments, an osteosarcoma is a metastatic osteosarcoma. In embodiments, an osteosarcoma is a MSI-H osteosarcoma. In embodiments, an osteosarcoma is a MSS osteosarcoma. In embodiments, an osteosarcoma is a POLE-mutant osteosarcoma. In embodiments, an osteosarcoma is a POLD-mutant osteosarcoma. In embodiments, an osteosarcoma is a high TMB osteosarcoma. In embodiments, an osteosarcoma is associated with homologous recombination repair deficiency/homologous repair deficiency (“HRD”). In embodiments, an osteosarcoma is a TP53 mutated osteosarcoma.

In embodiments, a cancer is a soft tissue sarcoma. In embodiments, a soft tissue sarcoma is an advanced soft tissue sarcoma. In embodiments, a soft tissue sarcoma is a metastatic soft tissue sarcoma. In embodiments, a soft tissue sarcoma is a MSI-H soft tissue sarcoma. In embodiments, a soft tissue sarcoma is a MSS soft tissue sarcoma. In embodiments, a soft tissue sarcoma is a POLE-mutant soft tissue sarcoma. In embodiments, a soft tissue sarcoma is a POLD-mutant soft tissue sarcoma. In embodiments, a soft tissue sarcoma is a high TMB soft tissue sarcoma. In embodiments, a soft tissue sarcoma is associated with homologous recombination repair deficiency/homologous repair deficiency (“HRD”). In embodiments, a soft tissue sarcoma is leiomyosarcoma. In embodiments, a soft tissue sarcoma is a TP53 mutated soft tissue sarcoma.

In embodiments, a cancer is Wilms tumor. In embodiments, Wilms tumor is an advanced Wilms tumor. In embodiments, Wilms tumor is a metastatic Wilms tumor. In embodiments, Wilms tumor is a MSI-H Wilms tumor. In embodiments, Wilms tumor is a MSS Wilms tumor. In embodiments, Wilms tumor is a POLE-mutant Wilms tumor. In embodiments, Wilms tumor is a POLD-mutant Wilms tumor. In embodiments, Wilms tumor is a high TMB Wilms tumor. In embodiments, Wilms tumor is associated with homologous recombination repair deficiency/homologous repair deficiency (“HRD”). In embodiments, Wilms tumor is a TP53 mutated Wilms tumor.

In embodiments, a cancer is cholangiocarcinoma.

In embodiments, a cancer is an unknown primary cancer.

In embodiments, a cancer is a gynecologic cancer. As used herein, a “gynecologic cancer” is a malignancy of the female reproductive tract, including, but not limited to, cervix, endometrium, fallopian tubes, ovaries, uterus, vagina, and breast.

In embodiments, the cancer is a gynecologic cancer selected from cervical cancer (e.g., squamous cell carcinoma, adenocarcinoma, adenosquamous carcinoma, small cell carcinoma), endometrial cancer (e.g., adenocarcinoma, sarcoma), ovarian/fallopian tube cancer (epithelial carcinoma, dysgerminoma, teratoma, endodermal sinus tumors, embryonal carcinoma, sex cord stromal tumors), gestational trophoblastic tumor, primary peritoneal cancer, uterine sarcoma, uterine papillary serous carcinoma, adnexal tumor, vaginal cancer (e.g., squamous cell carcinoma, adenocarcinoma, clear cell adenocarcinoma, melanoma, sarcoma), or vulvar cancer (squamous cell carcinoma, adenocarcinoma, melanoma, sarcoma, verrucous carcinoma).

In embodiments, the cancer is a breast cancer selected from ductal carcinoma in situ, lobular carcinoma, inflammatory breast cancer, invasive ductal carcinoma, Paget disease of the nipple, papillary breast cancer, medullary carcinoma, mammary carcinoma, PAM-50 classes (e.g., basal (triple-negative) cancer, HER2 positive cancer, luminal A cancer, luminal B cancer, normal-like cancer), or BRCA1 and/or BRCA2 mutation positive cancer. In embodiments, the breast cancer is a TP53 mutated breast cancer.

### TP53 Mutations

Tumor protein P53 (TP53), also known as cellular tumor antigen p53, and transformation-related protein 53 (TRP53), is a regulatory protein that is often mutated in human cancers. TP53 gene encodes proteins that bind to DNA and regulate gene expression to prevent mutations of the genome. In addition to the full-length protein, the human TP53 gene encodes at least 15 protein isoforms, all collectively referred to as “p53” or “p53 proteins”. p53 plays a role in regulation or progression through the cell cycle, apoptosis, and genomic stability. The p53 tumor suppressor protein (encoded by the TP53 gene) is a master transcription factor activated in response to cellular stress (e.g. DNA damage, heteroploidy or oncogenes), modulating core cellular response programs such as cell cycle arrest, apoptosis, senescence and variations in metabolism.

TP53 mutants may be a mutant of any isoform coding sequence, missense mutants, frameshift mutants, deletions, in-frame deletions, splice site mutants, or nonsense mutants. Mutant gene or mutant transcript sequences may be referred to as “variants” or “sequence variants”. TP53 mutations in tumor specimens (e.g. a patient biopsy) by various methods including whole exome sequencing (WES).

### PD-1 Antagonists

The present disclosure is directed to methods of treating and uses for treatment of cancer in a patient by administering a programmed cell death protein 1 (PD-1) antagonist. The PD-1 antagonists of the disclosure include biosimilars thereof.

As used herein, “PD-1 antagonist” means any chemical compound or biological molecule that blocks binding of programmed cell death ligand 1 (PD-L1) expressed on a cancer cell to PD-1 expressed on an immune cell (T cell, B cell or natural killer T (NKT) cell) and may also block binding of PD ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCDIL2, PDL2, B7-DC, Btdc and CD273 for PD-L2. Human PD-1 amino acid sequences can be found in NCBI Locus No.: NP 005009. Human PD-L1 and PD-L2 amino acid sequences can be found in NCBI Locus No.: NP 054862 and NP_079515, respectively.

PD-1 antagonists include a monoclonal antibody (mAb), or antigen binding fragment thereof, which specifically binds to PD-1 or PD-L1, for example, specifically binds to human PD-1 or human PD-L1. The mAb may be a human antibody, a humanized antibody or a chimeric antibody, and may include a human constant region. In some embodiments, the human constant region is selected from IgG1, IgG2, IgG3 or IgG4 constant regions, and in preferred embodiments, the human constant region is an IgG1 or IgG4 constant region. In some embodiments, the antigen binding fragment is selected from Fab, Fab′-SH, F(ab′) 2, scFv or Fv fragments.

In an embodiment, the PD-1 antagonist is an anti-PD-1 antibody. In an embodiment, the anti-PD-1 antibody is selected from dostarlimab or a biosimilar thereof, pembrolizumab or a biosimilar thereof, nivolumab or a biosimilar thereof, or cemiplimab or a biosimilar thereof. In an embodiment, the anti-PD-1 antibody is dostarlimab or a biosimilar thereof.

Dostarlimab is a PD-1-blocking IgG4 humanized monoclonal antibody commercially available as JEMPERLI. Dostarlimab is indicated for the treatment of adult patients with mismatch repair deficient (dMMR) recurrent or advanced: (i) endometrial cancer, as determined by an FDA-approved test, that has progressed on or following prior treatment with a platinum-containing regimen, or (ii) solid tumors, as determined by an FDA-approved test, that have progressed on or following prior treatment and who have no satisfactory alternative treatment options.

Pembrolizumab is a humanized monoclonal anti-PD-1 antibody commercially available as KEYTRUDA. Pembrolizumab is indicated for the treatment of some unresectable or metastatic melanomas. The amino acid sequence of pembrolizumab and methods of using are disclosed in U.S. Pat. No. 8,168,757. Pembrolizumab is indicated to treat some advanced endometrial cancers, typically after other treatments have been used. Pembrolizumab is indicated for the treatment of adult endometrial cancer patients wherein the cancer is a high level of microsatellite instability (MSI-H) or a defect in a mismatch repair gene (dMMR) cancer; or, if the cancer is characterized as having a high tumor mutational burden (TMB-H). A threshold for characterizing a cancer as TMB-His the occurrence of ≥10 mutations/megabase (mut/Mb) in the genome of a population of cancer cells in a biopsy obtained from a tumor or collection of cancer tissue.

Pembrolizumab can also be used along with the targeted drug lenvatinib (Lenvima) to treat advanced endometrial cancers that are not MMR deficient (dMMR) or MSI high (MSI-H), typically after at least one other drug treatment has been tried.

Nivolumab is a humanized monoclonal anti-PD-1 antibody commercially available as OPDIVO. Nivolumab is indicated for the treatment of some unresectable or metastatic melanomas. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in US Patent No. U.S. Pat. No. 8,008,449.

Cemiplimab is a human monoclonal anti-PD-1 antibody commercially available as LIBTAYO. Cemiplimab is indicated for the treatment of non-small cell lung cancer (NSCLC), basal cell carcinoma (BCC), and cutaneous squamous cell carcinoma (CSCC).

Exemplary antibody heavy chain and light chain sequences are provided in Table 1. In an embodiment, dostarlimab comprises the heavy and light chain sequences of SEQ ID NO: 1 and SEQ ID NO: 2, respectively. In an embodiment, an anti-PD-1 antibody comprises the heavy chain and light chain sequences of SEQ ID NO: 3 and SEQ ID NO: 4, respectively. In an embodiment, an anti-PD-1 antibody comprises the heavy chain and light chain sequences of SEQ ID NO: 5 and SEQ ID NO: 6, respectively. In an embodiment, an anti-PD-1 antibody comprises the heavy chain and light chain sequences of SEQ ID NO: 7 and SEQ ID NO: 8, respectively.

TABLE 1

| SEQ ID | | |
|---|---|---|
| NO | Name | Sequence |
| 1 | Dostarlimab | EVQLLESGGGLVQPGGSLRLSCAASGFTFSSYDMSWVRQ |
| | Heavy Chain | APGKGLEWVSTISGGGSYTYYQDSVKGRFTISRDNSKNT |
| | | LYLQMNSLRAEDTAVYYCASPYYAMDYWGQGTTVTVS |
| | | SASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFLGGPS |
| | | VFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHN |
| | | HYTQKSLSLSLGK |
| 2 | Dostarlimab | DIQLTQSPSFLSAYVGDRVTITCKASQDVGTAVAWYQQK |
| | Light Chain | PGKAPKLLIYWASTLHTGVPSRFSGSGSGTEFTLTISSLQP |
| | | EDFATYYCQHYSSYPWTFGQGTKLEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 3 | Pembrolizumab | QVQLVQSGVEVKKPGASVKVSCKASGYTFTNYYMYWV |
| | Heavy Chain | RQAPGQGLEWMGGINPSNGGTNFNEKFKNRVTLTTDSST |
| | | TTAYMELKSLQFDDTAVYYCARRDYRFDMGFDYWGQG |
| | | TTVTVSSASTKGPSVFPLAPCSRSTSESTAALGCLVKDYFP |
| | | EPVTVSWNSGALTSGVHTFPAVLQSSGLYSLSSVVTVPSS |
| | | SLGTKTYTCNVDHKPSNTKVDKRVESKYGPPCPPCPAPEF |
| | | LGGPSVFLFPPKPKDTLMISRTPEVTCVVVDVSQEDPEVQ |
| | | FNWYVDGVEVHNAKTKPREEQFNSTYRVVSVLTVLHQD |
| | | WLNGKEYKCKVSNKGLPSSIEKTISKAKGQPREPQVYTLP |
| | | PSQEEMTKNQVSLTCLVKGFYPSDIAVEWESNGQPENNY |
| | | KTTPPVLDSDGSFFLYSRLTVDKSRWQEGNVFSCSVMHE |
| | | ALHNHYTQKSLSLSLGK |
| 4 | Pembrolizumab | EIVLTQSPATLSLSPGERATLSCRASKGVSTSGYSYLHWY |
| | Light Chain | QQKPGQAPRLLIYLASYLESGVPARFSGSGSGTDFTLTISS |
| | | LEPEDFAVYYCQHSRDLPLTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 5 | Nivolumab | QVQLVESGGGVVQPGRSLRLDCKASGITFSNSGMHWVR |
| | Heavy Chain | QAPGKGLEWVAVIWYDGSKRYYADSVKGRFTISRDNSK |
| | | NTLFLQMNSLRAEDTAVYYCATNDDYWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFLGGPSVF |
| | | LFPPKPKDTLMISRTPEVTCVVVDVSQEDPEVQFNWYVD |
| | | GVEVHNAKTKPREEQFNSTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPV |
| | | LDSDGSFFLYSRLTVDKSRWQEGNVFSCSVMHEALHNH |
| | | YTQKSLSLSLGK |
| 6 | Nivolumab | EIVLTQSPATLSLSPGERATLSCRASQSVSSYLAWYQQKP |
| | Light Chain | GQAPRLLIYDASNRATGIPARFSGSGSGTDFTLTISSLEPED |
| | | FAVYYCQQSSNWPRTFGQGTKVEIKRTVAAPSVFIFPPSD |
| | | EQLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGEC |
| 7 | Cemiplimab | EVQLLESGGVLVQPGGSLRLSCAASGFTFSNFGMTWVRQ |
| | Heavy Chain | APGKGLEWVSGISGGGRDTYFADSVKGRFTISRDNSKNT |
| | | LYLQMNSLKGEDTAVYYCVKWGNIYFDYWGQGTLVTV |
| | | SSASTKGPSVFPLAPCSRSTSESTAALGCLVKDYFPEPVTV |
| | | SWNSGALTSGVHTFPAVLQSSGLYSLSSVVTVPSSSLGTK |
| | | TYTCNVDHKPSNTKVDKRVESKYGPPCPPCPAPEFLGGPS |
| | | VFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHN |
| | | HYTQKSLSLSLGK |
| 8 | Cemiplimab | DIQMTQSPSSLSASVGDSITITCRASLSINTFLNWYQQKPG |
| | Light Chain | KAPNLLIYAASSLHGGVPSRFSGSGSGTDFTLTIRTLQPED |
| | | FATYYCQQSSNTPFTFGPGTVVDFRRTVAAPSVFIFPPSDE |
| | | QLKSGTASVVCLLNNFYPREAKVQWKVDNALQSGNSQE |
| | | SVTEQDSKDSTYSLSSTLTLSKADYEKHKVYACEVTHQG |
| | | LSSPVTKSFNRGEC |

### Biosimilars

In some embodiments, the biosimilar biologic product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biologic product have been previously approved for the reference product. In some embodiments, the route of administration, the dosage form, and/or the strength of the biologic product are the same as those of the reference product. A “biosimilar” can be, e.g., a presently known antibody having the same or similar primary amino acid sequence as a marketed antibody (e.g., dostarlimab), but may be made in different cell types or by different production, purification, or formulation methods, or in a different formulation. In some embodiments the biosimilar biologic product is a biosimilar of dostarlimab. In some embodiments the biosimilar biologic product is a biosimilar of pembrolizumab. In some embodiments the biosimilar biologic product is a biosimilar of nivolumab.

In certain embodiments of the methods described herein, dostarlimab can be replaced with a biosimilar thereof. Accordingly, it will be understood that the term “dostarlimab” is intended to encompass any biosimilar(s) of dostarlimab. Also encompassed by the term “dostarlimab” are antibodies which have CDRs, variable regions, and/or heavy and light chains of dostarlimab. Additionally encompassed by the term “dostarlimab” are antibodies and/or any biosimilar(s) of dostarlimab which have CDRs, variable regions, and/or heavy and light chains that are at least 70%, at least 75%, at least 80%, at least 85%, at least 90%, at least 95%, at least 96%, at least 97%, at least 98%, or at least 99% identical to the CDRs, variable regions, or heavy and light chains, respectively, of dostarlimab.

In certain embodiments, encompassed by the term “dostarlimab” are antibodies and/or any biosimilar(s) of dostarlimab which comprise heavy chain and light chain sequences that are identical to those in the reference antibody (i.e., dostarlimab), except for having at least six, five, four, three, two, or one amino acid substitutions at positions that are located within and/or outside of the light chain CDRs and/or at least six, five, four, three, two, or one conservative amino acid substitutions that are located within and/or outside of the heavy chain CDRs (e.g., the variant positions are located in the framework regions or the constant region), and wherein the antibody or biosimilar is substantially the same as the reference antibody (i.e., dostarlimab) with respect to the following properties: binding affinity to the inhibitory checkpoint molecule (i.e., PD-1) and ability to block the binding of the inhibitory checkpoint molecule (i.e., PD-1) to its ligand (i.e., PD-L1 and/or PD-L2).

In certain embodiments, dostarlimab is a PD-1-blocking humanized antibody. In other embodiments, dostarlimab is a PD-1-blocking monoclonal antibody (mAb). In yet other embodiments, dostarlimab is a PD-1-blocking humanized mAb of the Ig4 kappa isotype. In specific embodiments, dostarlimab comprises a heavy chain comprising the amino acid sequence of SEQ ID NO:1, and a light chain comprising the amino acid sequence of SEQ ID NO:2. In certain aspects, dostarlimab comprises of two heavy and two light chains with a single N-linked glycosylation site on each heavy chain (“dostarlimab-gxly”). In certain embodiments, the single glycosylation site is at asparagine 293 on the heavy chain. In certain embodiments, dostarlimab comprises a heavy chain comprising the amino acid sequence of SEQ ID NO:1, and a light chain comprising the amino acid sequence of SEQ ID NO:2, comprising all functional post-translational modifications thereof. In some embodiments, dostarlimab has a calculated molecular weight of about 144 kDa.

The primary structure and function of dostarlimab are disclosed in International Publication Nos. WO 2014/179664, WO 2018/085468 and WO 2018/129559, each of which is incorporated by reference herein in its entirety.

### Chemotherapy

The PD-1 antagonists disclosed herein may be administered in combination with a chemotherapy regimen comprising one or more chemotherapeutic agents. Suitable chemotherapeutic agents include antimetabolite neoplastic agents (e.g., antifolate antineoplastic agents), platinum-based chemotherapy, and other known chemotherapeutic agents including, but not limited to, paclitaxel, docetaxel, and nab-paclitaxel (ABRAXANE).

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, capecitabine, cladribine, clofarabine, cytarabine, decitabine, floxuridine, fludarabine, fluorouracil, gemcitabine, hydroxyurea, mercaptopurine, methotrexate, nelarabine, pemetrexed, pralatrexate, and thioguanine.

Platinum-based chemotherapeutic agents induce cancer cell apoptosis as a response to their covalent binding to DNA. Examples of platinum-based agents include, but are not limited to, cisplatin, carboplatin, oxaliplatin, nedaplatin, triplatin tetranitrate, phenanthriplatin, picoplatin, and satraplatin.

In an embodiment, the one or more chemotherapeutic agents are selected from an antifolate antineoplastic agent, a platinum-based chemotherapy, paclitaxel, or combinations thereof.

In an embodiment, the antifolate antineoplastic agent is pemetrexed.

In an embodiment, the platinum-based chemotherapy is cisplatin. In an embodiment, the platinum-based chemotherapy is carboplatin.

In embodiments the PD-1 agents are administered in combination with a chemotherapy regimen. In embodiments the PD-1 agents are administered in combination with a radiotherapy regimen. In embodiments the PD-1 agents are administered in combination with both a chemotherapy regimen and a radiotherapy regimen.

In embodiments, the chemotherapy regimen is cisplatin followed by carboplatin and paclitaxel. In some embodiments a radiotherapy regimen is administered before carboplatin and paclitaxel.

In embodiments, a PD-1 antagonist regimen is administered after a chemotherapy regimen. In embodiments, a PD-1 antagonist regimen is administered in combination with a chemotherapy regimen and a radiotherapy regimen.

In embodiments, the chemotherapy regimen comprises one or more of the following: cisplatin and doxorubicin; cisplatin, doxorubicin and paclitaxel; cisplatin; carboplatin; doxorubicin; liposomal doxorubicin; paclitaxel; albumin-bound paclitaxel; topotecan; temsirolimus; cabozantinib; docetaxel; ifosfamide; ifosfamide and paclitaxel; cisplatin and ifosfamide.

### Doses and Dosing Regimens

### PD-1 Antagonists and Chemotherapeutic Agents

In some embodiments, the dose of the PD-1 antagonist (e.g., an anti-PD-1 antibody such as dostarlimab or a biosimilar thereof) is within a range of about 5 to about 5000 mg (e.g., about 5 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000 mg, about 3000 mg, about 4000 mg, about 5000 mg, or a range defined by any two of the foregoing values). In some embodiments, the dose of the PD-1 antagonist (e.g., an anti-PD-1 antibody such as dostarlimab or a biosimilar thereof) is 500 mg or 1000 mg.

In some embodiments, the dose is in an amount relative to body weight. In some embodiments, the dose of the PD-1 antagonist (e.g., an anti-PD-1 antibody such as dostarlimab) is within a range of about 0.01 mg/kg to 100 mg/kg of animal or human body weight; however, doses below or above this exemplary range are within the scope of the disclosure. The daily dose can be about 0.01 mg/kg to about 50 mg/kg of total body weight (e.g., about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, or a range defined by any two of the foregoing values).

In some embodiments, the dose is administered according to a regimen that delivers a dose every week. In some embodiments, the dose is administered according to a regimen that delivers a dose every two weeks. In some embodiments, the dose is administered according to a regimen that delivers a dose every three weeks. In some embodiments, the dose is administered according to a regimen that delivers a dose every four weeks. In some embodiments, the dose is administered according to a regimen that delivers a dose every six weeks. In some embodiments, the dose is administered according to a regimen that delivers a dose every eight weeks.

In some embodiments, the PD-1 antagonist (e.g., an anti-PD-1 antibody such as dostarlimab or a biosimilar thereof) is administered according to a regimen that delivers a first dose of the PD-1 antagonist for the first 2 to 6 dosing cycles (e.g., the first 3, 4, or 5 dosing cycles), and then delivers a second dose of the PD-1 antagonist for the subsequent dosing cycles until therapy is discontinued (e.g., due to disease progression or an adverse effect or as directed by a physician). In some embodiments, the duration of the first set of 2 to 6 dosing cycles (e.g., the first 3, 4, or 5 dosing cycles) is different from the duration of the subsequent dosing cycles. In some embodiments the first dose and the second dose are the same duration and/or amount. In some embodiments, the first dose and the second dose are different durations and/or amounts.

In an embodiment, the PD-1 antagonist is administered at a dose of 500 mg. In an embodiment, the PD-1 antagonist is administered through infusion Q3W. In an embodiment, the PD-1 antagonist is administered through a 30 minute infusion Q3W. In an embodiment, PD-1 antagonist is administered through infusion Q3W for up to three years. In an embodiment, PD-1 antagonist is administered through infusion Q3W until disease progression. In an embodiment, PD-1 antagonist is administered through infusion Q3W until a complete response.

In an embodiment, the PD-1 antagonist is administered at a dose of 1000 mg. In an embodiment, the PD-1 antagonist is administered through infusion Q6W. In an embodiment, the PD-1 antagonist is administered through a 30 minute infusion Q6W. In an embodiment, PD-1 antagonist is administered through infusion Q6W for up to three years. In an embodiment, PD-1 antagonist is administered through infusion Q6W until disease progression. In an embodiment, PD-1 antagonist is administered through infusion Q6W until a complete response.

In an embodiment, the pemetrexed is administered at a dose of 500 milligram per meter square (mg/m²). In an embodiment, the pemetrexed is administered Q3W. In an embodiment, the pemetrexed is administered through a 10 minute IV infusion Q3W.

In an embodiment, the cisplatin is administered at a dose of 75 mg/m². In an embodiment, the cisplatin is administered Q3W. In an embodiment, the cisplatin is administered through a 30 minute IV infusion Q3W for 4 to 6 cycles, such as 6 cycles.

In some embodiments, the PD-1 antagonist is administered at a dose of 200 mg every 3 weeks or at a dose of 400 mg every 6 weeks.

In an embodiment, the carboplatin is administered at a dose at an area under the concentration time curve of 5 milligram/milliliters/minute (mg/mL/min). In an embodiment, the carboplatin is administered Q3W. In an embodiment, the carboplatin is administered through a 15 to 60 minute IV infusion Q3W for 4 to 6 cycles, such as 6 cycles.

In an embodiment, the paclitaxel is administered at a dose of 175 milligram per meter square (mg/m²). In an embodiment, the paclitaxel is administered Q3W. In an embodiment, the paclitaxel is administered Q3W for 4 to 6 cycles, such as 6 cycles.

### Dostarlimab and Optional Chemotherapeutic Agents

In some embodiments, the dose of dostarlimab or a biosimilar thereof is within a range of about 5 to about 5000 mg (e.g., about 5 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000 mg, about 3000 mg, about 4000 mg, about 5000 mg, or a range defined by any two of the foregoing values). In some embodiments, the dose dostarlimab or a biosimilar thereof is 500 mg or 1000 mg.

In some embodiments, the dose is in an amount relative to body weight. In some embodiments, the dose of dostarlimab or a biosimilar thereof is within a range of about 0.01 mg/kg to 100 mg/kg of animal or human body weight; however, doses below or above this exemplary range are within the scope of the disclosure. The daily dose can be about 0.01 mg/kg to about 50 mg/kg of total body weight (e.g., about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, or a range defined by any two of the foregoing values).

In some embodiments, the dose is administered according to a regimen that delivers a dose every two weeks. In some embodiments, the dose is administered according to a regimen that delivers a dose every three weeks. In some embodiments, the dose is administered according to a regimen that delivers a dose every four weeks. In some embodiments, the dose is administered according to a regimen that delivers a dose every six weeks. In some embodiments, the dose is administered according to a regimen that delivers a dose every eight weeks.

In some embodiments, a composition that delivers dostarlimab or a biosimilar thereof is administered to a patient at a dose of about 400 mg. In some embodiments, dostarlimab or a biosimilar thereof is administered according to a regimen that delivers a dose of about 400 mg every two weeks. In some embodiments, dostarlimab or a biosimilar thereof is administered according to a regimen that delivers a dose of about 400 mg every three weeks. In some embodiments, dostarlimab or a biosimilar thereof is administered according to a regimen that delivers a dose of about 400 mg every four weeks. In some embodiments, dostarlimab or a biosimilar thereof is administered according to a regimen that delivers a dose of about 400 mg every six weeks. In some embodiments, dostarlimab or a biosimilar thereof is administered according to a regimen that delivers a dose of about 400 mg every eight weeks.

In some embodiments, a composition that delivers dostarlimab or a biosimilar thereof is administered to a patient at a dose of about 500 mg. In some embodiments, dostarlimab or a biosimilar thereof is administered according to a regimen that delivers a dose of about 500 mg every two weeks. In some embodiments, dostarlimab or a biosimilar thereof is administered according to a regimen that delivers a dose of about 500 mg every three weeks. In some embodiments, dostarlimab or a biosimilar thereof is administered according to a regimen that delivers a dose of about 500 mg every four weeks. In some embodiments, dostarlimab or a biosimilar thereof is administered according to a regimen that delivers a dose of about 500 mg every six weeks. In some embodiments, dostarlimab or a biosimilar thereof is administered according to a regimen that delivers a dose of about 500 mg every eight weeks.

In some embodiments, a composition that delivers dostarlimab or a biosimilar thereof is administered to a patient at a dose of about 800 mg. In some embodiments, dostarlimab or a biosimilar thereof is administered according to a regimen that delivers a dose of about 800 mg every two weeks. In some embodiments, dostarlimab or a biosimilar thereof is administered according to a regimen that delivers a dose of about 800 mg every three weeks. In some embodiments, dostarlimab or a biosimilar thereof is administered according to a regimen that delivers a dose of about 800 mg every four weeks. In some embodiments, dostarlimab or a biosimilar thereof is administered according to a regimen that delivers a dose of about 800 mg every six weeks. In some embodiments, dostarlimab or a biosimilar thereof is administered according to a regimen that delivers a dose of about 800 mg every eight weeks.

In some embodiments, a composition that delivers dostarlimab or a biosimilar thereof is administered to a patient at a dose of about 1,000 mg. In some embodiments, dostarlimab or a biosimilar thereof is administered according to a regimen that delivers a dose of about 1,000 mg every two weeks. In some embodiments, dostarlimab or a biosimilar thereof is administered according to a regimen that delivers a dose of about 1,000 mg every three weeks. In some embodiments, dostarlimab or a biosimilar thereof is administered according to a regimen that delivers a dose of about 1,000 mg every four weeks. In some embodiments, dostarlimab or a biosimilar thereof is administered according to a regimen that delivers a dose of about 1,000 mg every six weeks. In some embodiments, dostarlimab or a biosimilar thereof is administered according to a regimen that delivers a dose of about 1,000 mg every eight weeks.

In some embodiments, dostarlimab or a biosimilar thereof is administered according to a regimen that delivers a dose of about 500 mg every three weeks. In some embodiments, dostarlimab or a biosimilar thereof is administered according to a regimen that delivers a dose of about 1000 mg every six weeks.

In some embodiments, dostarlimab or a biosimilar thereof is administered according to a regimen that delivers a first dose of dostarlimab or a biosimilar thereof for the first 2 to 6 dosing cycles (e.g., the first 3, 4, or 5 dosing cycles), and then delivers a second dose of dostarlimab or a biosimilar thereof for the subsequent dosing cycles until therapy is discontinued (e.g., due to disease progression or an adverse effect or as directed by a physician). In some embodiments, the duration of the first set of 2 to 6 dosing cycles (e.g., the first 3, 4, or 5 dosing cycles) is different from the duration of the subsequent dosing cycles. In embodiments, dostarlimab or a biosimilar thereof is administered according to a regimen that delivers a first dose of dostarlimab or a biosimilar thereof once every three weeks for the first three dosing cycles, and then delivers a second dose of dostarlimab or a biosimilar thereof once every six weeks or more for the remaining dosing cycles (e.g., a second dose of dostarlimab or a biosimilar thereof once every six weeks for the remaining dosing cycles). In embodiments, dostarlimab or a biosimilar thereof is administered according to a regimen that delivers dostarlimab or a biosimilar thereof once every three weeks for the first four dosing cycles, and then delivers a second dose of dostarlimab or a biosimilar thereof once every six weeks or more for the remaining dosing cycles (e.g., a second dose of dostarlimab or a biosimilar thereof once every six weeks for the remaining dosing cycles). In embodiments, dostarlimab or a biosimilar thereof is administered according to a regimen that delivers a first dose of dostarlimab or a biosimilar thereof once every three weeks for the first five dosing cycles, and then delivers a second dose of a dostarlimab or a biosimilar thereof once every six weeks or more for the remaining dosing cycles (e.g., a second dose of a dostarlimab or a biosimilar thereof once every six weeks for the remaining dosing cycles). In embodiments, dostarlimab or a biosimilar thereof is administered according to a regimen that delivers dostarlimab or a biosimilar thereof once every three weeks for the first six dosing cycles, and then delivers a second dose of dostarlimab or a biosimilar thereof once every six weeks or more for the remaining dosing cycles (e.g., a second dose of dostarlimab or a biosimilar thereof once every six weeks for the remaining dosing cycles).

In some embodiments, a dostarlimab or a biosimilar thereof is administered according to a regimen that delivers a first dose of dostarlimab or a biosimilar thereof once every three weeks for the first 2 to 6 dosing cycles (e.g., the first 3, 4, or 5 dosing cycles), and then delivers a second dose of dostarlimab or a biosimilar thereof once every six weeks or until therapy is discontinued (e.g., due to disease progression or an adverse effect or as directed by a physician). In some embodiments, a dostarlimab or a biosimilar thereof is administered according to a regimen that delivers a first dose of dostarlimab or a biosimilar thereof once every three weeks for the first 3, 4, 5, or 6 dosing cycles (e.g., the first 4 dosing cycles or the first 6 dosing cycles), and then delivers a second dose of dostarlimab or a biosimilar thereof once every six weeks or more until therapy is discontinued (e.g., due to disease progression or an adverse effect or as directed by a physician). In embodiments, the method comprises delivering a second dose of dostarlimab or a biosimilar thereof once every six weeks until therapy is discontinued.

In some embodiments the first and/or second dose of dostarlimab or a biosimilar thereof is about 100 mg to about 2,000 mg (e.g., about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg). In some embodiments the first dose and the second dose are the same. In some embodiments, the first dose and the second dose are different. In embodiments, the first dose is about 500 mg of dostarlimab or a biosimilar thereof. In embodiments, the second dose is about 1000 mg of dostarlimab or a biosimilar thereof.

In some embodiments, dostarlimab or a biosimilar thereof is administered according to a regimen that comprises administering about 500 mg dose every 3 weeks for four doses followed by administering at least one about 1,000 mg dose every six weeks after the fourth dose of about 500 mg. In some embodiments, dostarlimab or a biosimilar thereof is administered according to a regimen that comprises administering about 500 mg dose every 3 weeks for six doses followed by administering at least one about 1,000 mg dose every six weeks after the sixth dose of about 500 mg. In some embodiments, additional about 1,000 mg doses are administered every six weeks after the first about 1000 mg dose until no further clinical benefit is achieved. In some particular embodiments, dostarlimab or a biosimilar thereof is administered according to a dosing regimen that includes 500 mg for 4 cycles Q3W followed by 1000 mg Q6W. In some particular embodiments, dostarlimab or a biosimilar thereof is administered according to a dosing regimen that includes 500 mg for 6 cycles Q3W followed by 1000 mg Q6W.

In an embodiment, the dostarlimab or a biosimilar thereof is administered at a dose of 500 mg. In an embodiment, the dostarlimab or a biosimilar thereof is administered Q3W. In an embodiment, the dostarlimab or a biosimilar thereof is administered through a 30 minute infusion intravenously Q3W. In an embodiment, the dostarlimab or a biosimilar thereof is administered at a dose of 1,000 mg beginning 3 weeks after dose 4. In an embodiment, the dostarlimab or a biosimilar thereof is administered at a dose of 1,000 mg beginning 3 weeks after dose 6. In an embodiment, the dostarlimab or a biosimilar thereof is administered every 6 weeks.

In an embodiment, the patient discontinued administration of at least one of the one or more chemotherapeutic agents and/or radiation therapy prior to beginning administration of dostarlimab or a biosimilar thereof.

### Other Combination Therapy

In some embodiments, the dose of the PD-1 antagonist (e.g., an anti-PD-1 antibody such as dostarlimab or a biosimilar thereof) In an embodiment, the dostarlimab or a biosimilar thereof is administered at a dose of 500 mg. In an embodiment, the dostarlimab or a biosimilar thereof is administered Q3W. In an embodiment, the dostarlimab or a biosimilar thereof is administered through a 30 minute infusion intravenously Q3W. In an embodiment, the dostarlimab or a biosimilar thereof is administered at a dose of 1,000 mg beginning 3 weeks after dose 4. In an embodiment, the dostarlimab or a biosimilar thereof is administered at a dose of 1,000 mg beginning 3 weeks after dose 6. In an embodiment, the dostarlimab or a biosimilar thereof is administered every 6 weeks. In some embodiments, the PD-1 antagonist is administered with lenvatinib 20 mg orally once daily.

In some embodiments, the PD-1 antagonist is pembrolizumab or a biosimilar thereof. In an embodiment, the pembrolizumab or a biosimilar thereof is administered at a dose of 200 mg every three weeks. In an embodiment, the pembrolizumab or a biosimilar thereof is administered at a dose of 400 mg every six weeks. In some embodiments, the pembrolizumab or a biosimilar is administered with lenvatinib 20 mg orally once daily.

In an embodiment, the patient discontinued administration of at least one of the one or more chemotherapeutic agents and/or radiation therapy prior to beginning administration of the PD-1 antagonist. In an embodiment the PD-1 antagonist is dostarlimab or a biosimilar thereof. In an embodiment the PD-1 antagonist is pembrolizumab or a biosimilar thereof

### Measuring Tumor Response

Tumor response can be measured by, for example, the Response Evaluation Criteria in Solid Tumors (RECIST) v1.1 guidelines. The guidelines are provided by E. A. Eisenhauer, et al, “New response evaluation criteria in solid tumors: Revised RECIST guideline (version 1.1.),” Eur. J. of Cancer, 45:228-247 (2009). In some embodiments, RECIST guidelines may serve as a basis for all protocol guidelines related to disease status. In some embodiments, RECIST guidelines are used to assess tumor response to treatment and/or date of disease progression.

RECIST guidelines require, first, estimation of the overall tumor burden at baseline, which is used as a comparator for subsequent measurements. Tumors can be measured via use of any imaging system known in the art, for example, by a CT scan, or an X-ray. Measurable disease is defined by the presence of at least one measurable lesion.

The following examples are provided to illustrate, but not limit the claimed disclosure.

### EXAMPLES

### Example 1: Phase 3 Study of Dostarlimab Plus Standard of Care Chemotherapy Versus Placebo Plus Standard of Care Chemotherapy in Patients with Primary Advanced or Recurrent Endometrial Cancer

A phase 3 study was conducted to compare the efficacy of dostarlimab plus carboplatin-paclitaxel followed by dostarlimab to carboplatin-paclitaxel plus placebo followed by placebo in patients with primary advanced or recurrent endometrial cancer. The primary endpoints were progression-free survival (PFS), per RECIST v1.1 by investigator assessment, and overall survival (OS). The secondary endpoints included PFS per blinded independent central review (BICR), overall response rate (ORR), duration of response (DoR), disease control rate (DCR), patient-reported outcomes (PROs), and safety and tolerability.

Patients in the experimental arm received dostarlimab, carboplatin, and paclitaxel Q3W for 6 cycles, followed by dostarlimab Q6W for up to 3 years. Patients in the control arm received placebo, carboplatin, and paclitaxel Q3W for 6 cycles, followed by placebo Q6W for up to 3 years. Dostarlimab was administered intravenously at a dose of 500 mg Q3W for 6 cycles, followed by a dose of 1000 mg Q6W for up to 3 years. Carboplatin was administered at an area under the concentration time curve of 5 milligram/milliliters/minute (mg/mL/min) Q3W for 6 cycles. Paclitaxel was administered at a dose of 175 milligram per meter square (mg/m²) Q3W for 6 cycles.

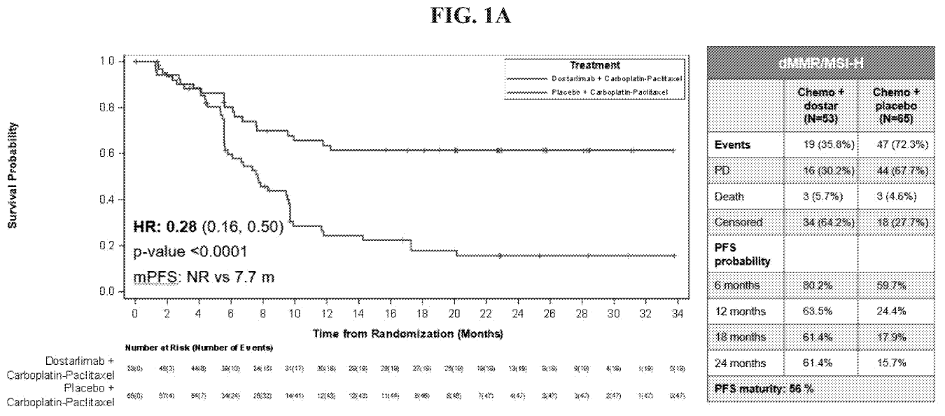

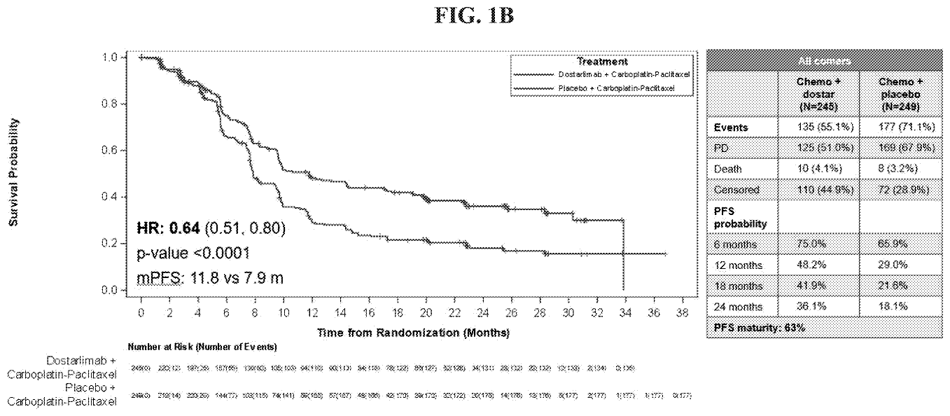

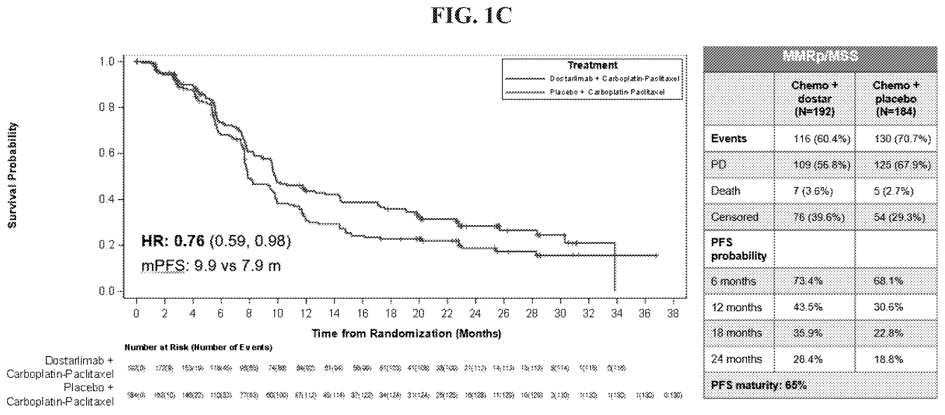

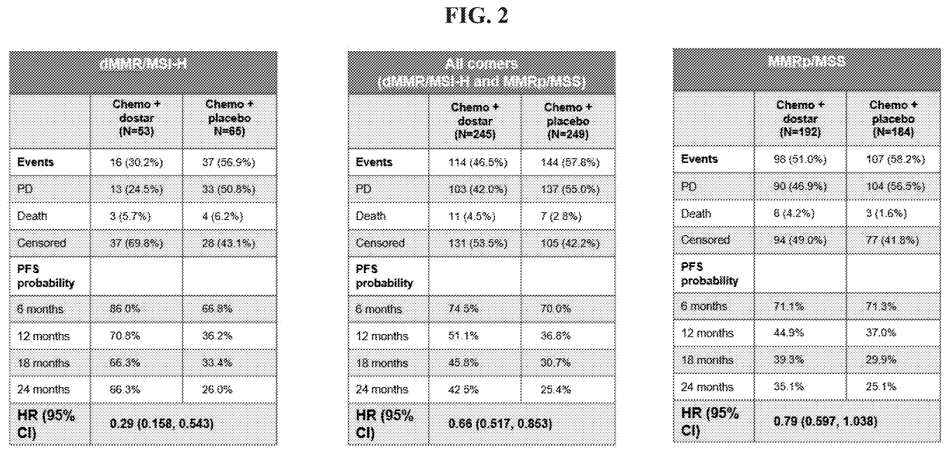

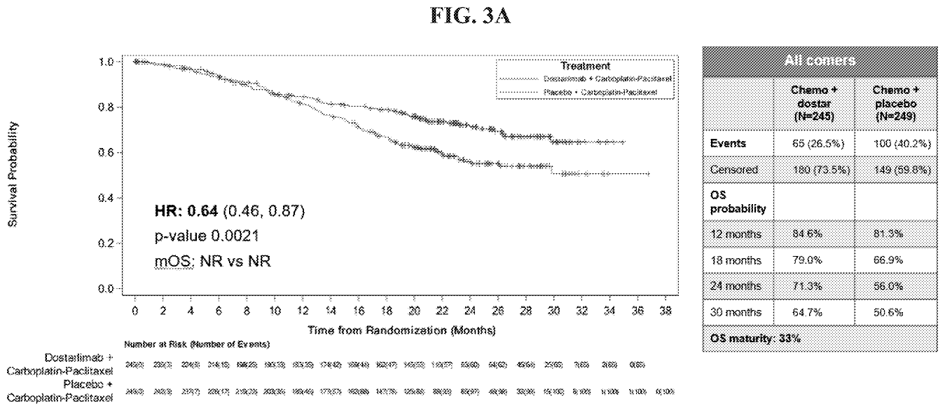

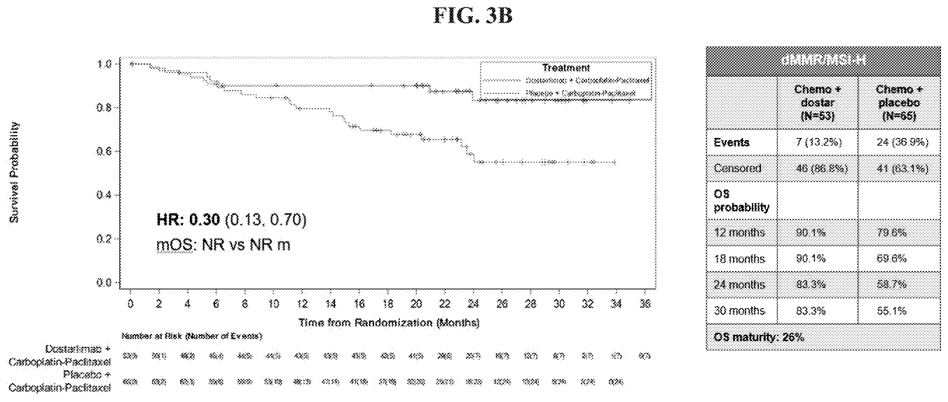

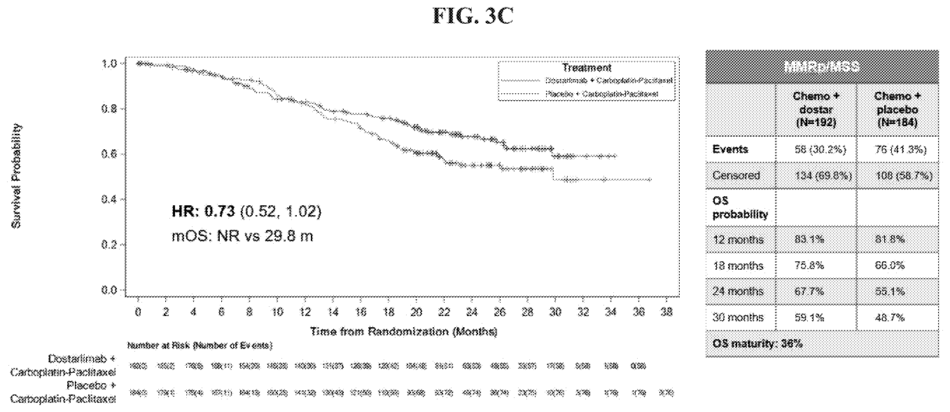

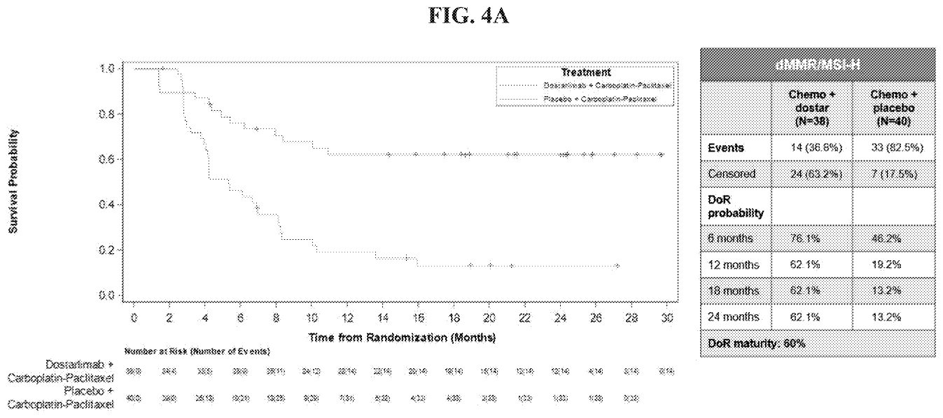

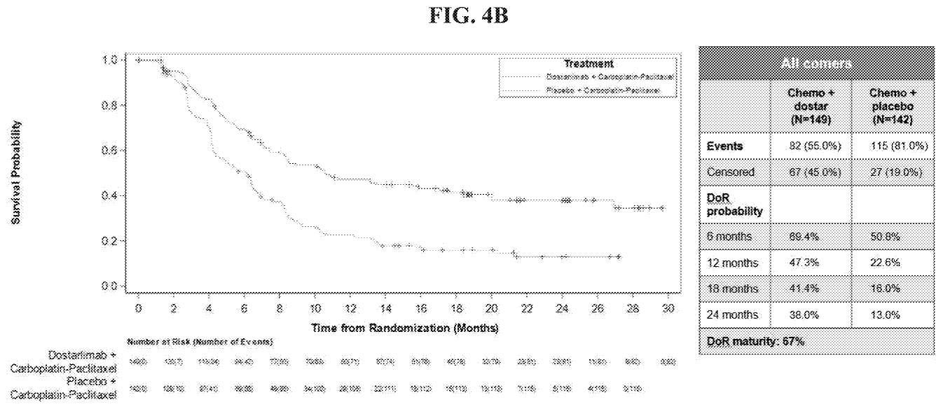

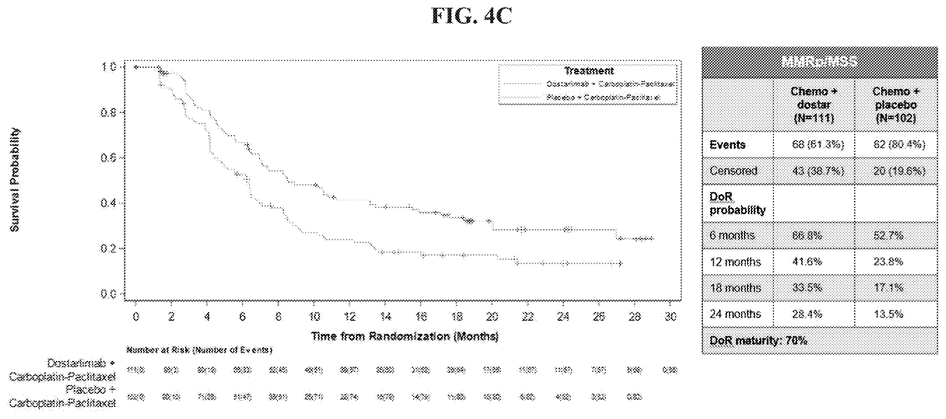

Surprisingly and unexpectedly, dostarlimab plus carboplatin-paclitaxel followed by dostarlimab significantly improved progression-free survival compared to carboplatin-paclitaxel plus placebo followed by placebo as shown in FIG. 1 and FIG. 2. Additionally, administration of dostarlimab plus carboplatin-paclitaxel followed by dostarlimab demonstrated a statistically significant and clinically meaningful benefit in a mismatch repair deficient (dMMR)/microsatellite instability-high (MSI-H) subgroup as shown in FIGS. 1A and 1n the overall population of adult patients with primary advanced or recurrent endometrial cancer as shown in FIG. 1B. A clinically relevant benefit in PFS was also observed in the mismatch repair proficient (MMRp)/microsatellite stable (MSS) subgroup as shown in FIG. 1C. In addition, favorable overall survival data across all subgroups was observed as shown in FIG. 3. Dostarlimab plus chemotherapy led to deeper and more durable responses vs. chemotherapy alone, with higher complete responses and longer duration of response as shown in FIG. 4.

Patient endometrial cancer history is provided in Table 2.

TABLE 2

| | dMMR/MSI-H | | | | All-comers | | | |
|---|---|---|---|---|---|---|---|---|
| | Dostarlimab + | | Placebo + | | Dostarlimab + | | Placebo + | |
| Variable | Chemo | | Chemo | | Chemo | | Chemo | |
| Reason [n (%)] | (N = 53) | | (N = 65) | | (N = 245) | | (N = 249) | |
| FIGO Stage at Diagnosis | | | | | | | | |
| Stage I | 18 | (34.0%) | 22 | (33.8%) | 65 | (26.5%) | 71 | (28.5%) |
| Stage II | 3 | (5.7%) | 5 | (7.7%) | 13 | (5.3%) | 13 | (5.2%) |
| Stage III | 14 | (26.4%) | 20 | (30.8%) | 75 | (30.6%) | 65 | (26.1%) |
| Stage IV | 14 | (26.4%) | 15 | (23.1%) | 72 | (29.4%) | 84 | (33.7%) |
| Unknown | 4 | (7.5%) | 3 | (4.6%) | 20 | (8.2%) | 16 | (6.4%) |
| Histology at Diagnosis | | | | | | | | |
| Carcinosarcoma | 4 | (7.5%) | 1 | (1.5%) | 25 | (10.2%) | 19 | (7.6%) |
| Endometroid | 44 | (83.0%) | 56 | (86.2%) | 134 | (54.7%) | 136 | (54.6%) |
| Mixed Carcinoma ≥10% | 2 | (3.8%) | 4 | (6.2%) | 10 | (4.1%) | 9 | (3.6%) |
| of carcinosarcoma, clear | | | | | | | | |
| cell or serous histology | | | | | | | | |
| Serous adenocarcinoma | 1 | (1.9%) | 1 | (1.5%) | 50 | (20.4%) | 52 | (20.9%) |
| Clear cell | 0 | | 0 | | 8 | (3.3%) | 9 | (3.6%) |
| adenocarcinoma | | | | | | | | |
| Mucinous | 0 | | 0 | | 0 | | 1 | (0.4%) |
| adenocarcinoma | | | | | | | | |
| Undifferentiated | 0 | | 0 | | 1 | (0.4%) | 2 | (0.8%) |
| carcinoma | | | | | | | | |
| Other | 2 | (3.8%) | 3 | (4.6%) | 17 | (6.9%) | 21 | (8.4%) |

The safety and tolerability profile of dostarlimab was consistent with clinical trials of similar regimens. The most common treatment-emergent adverse events in patients receiving dostarlimab plus chemotherapy were nausea, alopecia, fatigue, peripheral neuropathy, anemia, arthralgia, constipation, and diarrhea.

Whole exome sequencing (WES) was performed on the submitted patient biopsy samples. DNA isolation was performed on formalin-fixed paraffin-embedded (FFPE) archival or freshly collected baseline biopsy samples from RUBY Part 1 patients and checked for quality. Following quality check, whole exome sequencing was performed on isolated DNA using the Personalis ImmunoID Next© assay which allows for identification of a spectrum of variant types: single nucleotide variants (SNVs), short insertions and deletions (indels), copy number alterations (CNAs), and gene fusions. Bioinformatic analysis of raw sequencing data allowed for identification of POLE-EDM mutations and TP53 mutations. These mutation calls, combined with MMR/MSI status available from all patients enrolled on RUBY Part 1, allowed for assignment of each individual patient into a specific molecular subgroup.

The Personalis variant calling pipeline identified SNVs and other alterations in TP53 in RUBY Part 1 patients. Since there were no matched normal samples, the Personalis WES variant calling pipeline was run by the vendor in “Tumor-only” mode, where individual variants must exceed a 5% variant allele fraction threshold to be included into the summary variant reports. Further, all 5′ UTR, 3′ UTR, and upstream/downstream variants were filtered out, as they are not likely to have high functional impact on either POLE or TP53.

Identification of functional POLE EDM mutations: POLE variants in RUBY were identified by selecting only pathogenic mutations in the POLE exonuclease domain (EDM). Pathogenic set of POLE EDM mutations were derived from Table 3 of Leon-Castillo (Leon-Castillo, A., et al., J Pathol, 2020. 250 (3): p. 323-335) and this same set is also referenced in the ESMO guidelines for EC molecular classification (Oaknin, A., et al., Ann Oncol, 2022. 33 (9): p. 860-877) (Table 3).

TABLE 3
POLE missense variant set used for classification.

| | |
|---|---|
| POLE reference set (N = 11 | P286R, V411L, S297F, A456P, |
| missense variants) associated | S459F, F367S, L424I, M295R, |
| with POLE EDM disruption | P436R, M444K, D368Y |

Identification of functional TP53 mutations: We used a conservative approach for identification of TP53 mutations with functional consequences, in order to avoid misclassification of benign TP53 polymorphisms as TP53 mutations. First, likely TP53 germline variants from the list of missense variants were filtered using a gnomAD MAF <0.01 threshold, which removes variants that are seen at greater than 1% frequency in the human population based on the gnomAD database. Any detected frameshift, nonsense (truncating), and splice site variants were included as “pathogenic”. Any detected missense mutations in RUBY were called “pathogenic” if they were present in the reference missense mutation set (N=32) of cbioportal+clinvar (Table 4 A). This reference missense mutation set (N=32) was determined by using publicly available somatic mutation dataset to identify mutations previously observed in endometrial cancer, as well as having evidence that they can cause Li-Fraumeni or other hereditary TP53-driven cancers (ClinVar submissions). Therefore, publicly available mutational datasets with associated functional annotations for endometrial cancer were identified on the Cbioportal (www.cbioportal.org) comprising 12 studies (n=2214 samples; n=2204 unique patients) (Cerami, E., et al., Cancer Discov, 2012. 2 (5): p. 401-4; Gao, J., et al.,. Sci Signal, 2013. 6 (269): p. 11). Upon querying the TP53 gene within this dataset, the subset of unique samples with the presence of a TP53 alteration (n=495) was downloaded for analysis. We further selected only for missense mutations annotated as “pathogenic” by Clin Var (based on germline evidence in Li-Fraumeni syndrome and other hereditary cancer-predisposing syndromes) (Landrum, M. J., et al., Nucleic Acids Res, 2018. 46 (D1): p. D1062-D1067).

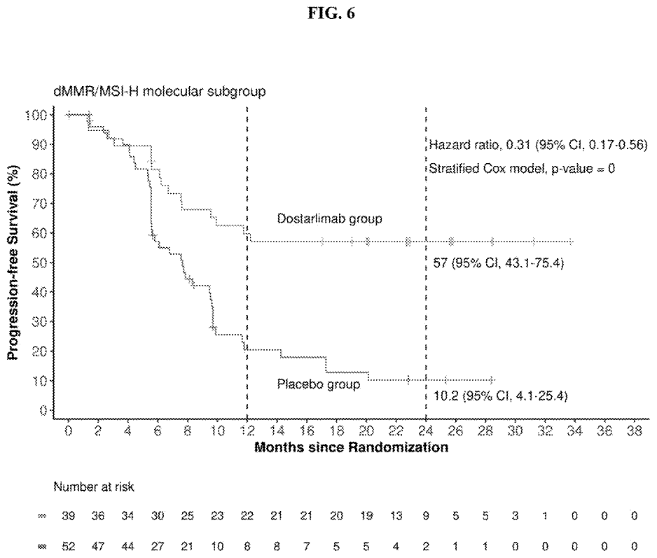

Assignment of molecular EC subgroups for RUBY Part 1: The molecular classification algorithm, as described in Oaknin et al. was used to group patients first into the POLE-mutated (POLE-mut) subtype, The POLE-wild type patients were then grouped into the dMMR/MSI-H subtype, using the RUBY part 1 source data verified MMR/MSI result. Finally, the MMRp/MSS patients were grouped into either the TP53-mut (TP53 mutated as described above) subgroup or NSMP (TP53-wildtype) (FIG. 6).

Comparing TP53 mutation prevalence in serous histology endometrial cancer for RUBY Part 1 to PanCan Atlas.: To test whether our TP53-mut classifier yields a similar distribution in serous histology EC cases between RUBY Part 1 and publicly available data, data for a total of N=115 patients with serous endometrial adenocarcinoma, with or without annotated TP53 alterations, was downloaded from the PanCan atlas portal (https://gdc.cancer.gov/about-data/publications/pancanatlas Mutations dataset “mc3.v0.2.8.PUBLIC.maf.gz”) [11]. We used assessed the prevalence of TP53 mutations in the N=115 specimens from the serous endometrial adenocarcinoma case by counting only pathogenic missense TP53 mutations, as provided by PanCan Atlas in the ClinVar significance field, and including frameshift, insertion/deletion, nonsense mutations and splice site variants. As such, the TP53 mutation prevalence in serous histology RUBY part 1 can be compared to PanCan Atlas N=115 serous endometrial adenocarcinomas, as similar criteria were used for calling functional missense TP53 mutations.

### Primary Endpoint Analysis

### Stratified Cox Proportional Hazards Model and Stratified Logrank Tests

The comparison of the distribution of PFS per the investigator and OS between the two treatment groups was performed using a stratified Cox proportional hazards model for each of the 4 molecular subgroups as defined above. The source-verified stratification factors of prior external pelvic radiotherapy (yes or no) and disease status (recurrent, primary Stage III, or primary Stage IV) were used as the strata. Censoring rules for primary analysis of PFS and OS as specified in the clinical SAP amendment 1 were used.

The hazard ratio with two-sided 95% CI was derived from the stratified Cox proportional hazards model and a two-sided p-value was calculated. In this analysis, the baseline hazard function was allowed to vary across strata (i.e., the two stratification variables of: source-verified prior pelvic radiotherapy and disease status).

Two-sided nominal p-values are presented. No adjustments for multiple comparisons were performed. These post-hoc exploratory analyses were evaluated using a 5% significance level.

Separately, stratified logrank tests between the two treatments were performed for each of the dMMR-MSI-H, TP53-mut, and NSMP molecular subgroups. The stratified logrank tests used strata as specified above, two-sided nominal p-values, and a 5% significance level.

### Kaplan-Meier (K-M) Estimates

The Kaplan-Meier estimates of the PFS and OS distribution function were computed and plotted graphically for each treatment group. The median PFS for each treatment group was provided along with the approximate 95% CIs. Results at the 25% and 75% percentiles were provided. The survival probabilities at 24 months and their associated 95% CIs were summarized by treatment group for each molecular subtype.

### Results

The WES data were processed with the Personalis variant (mutant) calling pipeline results and intersected the detected missense variants with the POLE EDM reference set of missense variants. This identified N=2 subjects with P286R and N=3 subjects with V411L, for a total of N=5 POLE-mutated subjects. Note, in N=1 specimen both V411L and S297P missense mutations were identified, and that specimen is classified as POLE-mut based on V411L alone, however notably the S297P mutation occurs at the same amino acid position as the S297F variant that is part of the POLE reference set.

We processed Cbioportal EC TP53 mutational data with Clin Var “pathogenic” annotations, identifying a set of N=32 missense mutations to use as a final reference set of missense mutations in TP53 that are likely associated with TP53 protein dysfunction (Table 4 A, “primary analysis reference set”).

To determine which samples from RUBY Part 1 had pathogenic TP53 mutations, the Personalis variant calling pipeline detected a total of N=156 missense variants, N=23 frameshift variants, N=2 in-frame deletions, N=11 splice site variants, and N=9 nonsense variants. TP53 missense variants that were identified by the Personalis variant calling pipeline but were not present in the “primary analysis reference set” (Table 4 A) were filtered out. This resulted in a final set of N=55 missense variants, N=23 frameshift variants, N=2 in-frame deletions, N=11 splice site variants, and N=9 nonsense variants that were used for molecular classification. This final set identified a total of N=96 RUBY Part 1 specimens with TP53 mutations that are highly likely to be associated with TP53 dysfunction.

TABLE 4
TP53 Missense variant sets used for mutant classification.

| | |
|---|---|
| A) Primary analysis | A276G, C141Y, C238F, C238R, C238W, D281G, D281N, D281Y, |
| reference set (N = 32 | G244S, G245S, G245V, H193P, I195F, I195T, P151R, P152L, R110P, |
| missense variants) | R158G, R175H, R213Q, R248P, R248Q, R248W, R273H, R273L, |
| | R282G, S215I, S241Y, V173M, Y163C, Y220C, Y220S |
| B) Sensitivity | A138P, A159D, A159V, A276G, C135F, C135R, C141Y, C176F, |
| analysis reference set | C176Y, C238F, C238G, C238R, C238S, C238W, C238Y, C242Y, |
| (N = 140 missense | C275F, C275Y, D281G, D281N, D281V, D281Y, E171D, E171G, |
| variants) | E258K, E285K, E285V, E286K, F134I, F270S, G105C, G105R, |
| | G105S, G244A, G244D, G244S, G245A, G245C, G245D, G245S, |
| | G245V, G266E, G266R, H168L, H179D, H179Q, H179Y, H193P, |
| | H193R, H193Y, I232N, I232S, I232T, I251L, I254N, I254T, K132E, |
| | K132N, K132R, L130H, L130P, L145Q, L194F, L265P, L35F, |
| | M133T, M237I, M246I, M246V, N131I, N131Y, P151A, P151R, |
| | P151S, P151T, P152L, P152R, P177R, P278T, R110L, R110P, R158G, |
| | R158H, R158L, R158S, R175G, R175H, R175L, R181P, R213P, |
| | R213Q, R248G, R248L, R248P, R248Q, R248W, R249T, R267G, |
| | R267W, R273C, R273G, R273H, R273L, R273P, R273S, R280S, |
| | R280T, R282G, R282P, R282W, R337C, R337H, R342P, S127C, |
| | S127P, S127Y, S215I, S240G, S241A, S241C, S241F, S241Y, T125K, |
| | T125R, T253A, V157A, V173A, V173L, V173M, V272L, V272M, |
| | Y163C, Y205C, Y220C, Y220H, Y220S, Y234C, Y234H, Y234N, |
| | Y236H |

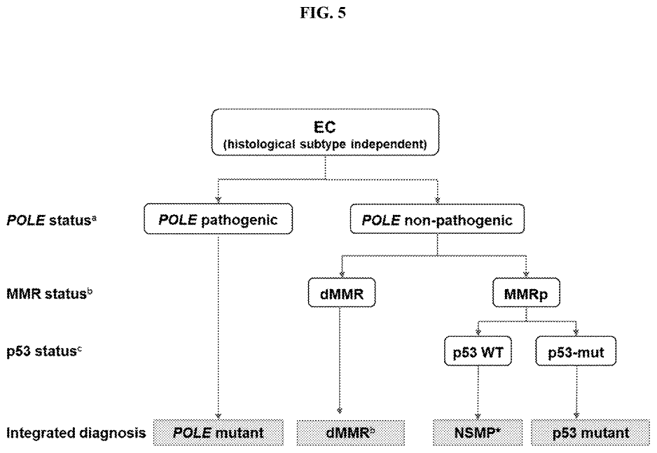

Out of a total of 494 participants enrolled in RUBY Part 1, WES results were available from a large majority of the patient specimens (N=400), which were then assigned to individual molecular subgroups as described in Methods 3.2c (FIG. 5).

Following the molecular classification algorithm, based on the detected POLE and TP53 mutations in WES data and the source data verified dMMR or MSI-H calls, we categorized N=400 RUBY specimens into: N=5 POLE-mut, N=91 dMMR/MSI-H, N=88 TP53-mut, and N=216 NSMP. Of note, there were no specimens with both POLE and TP53 mutations and there were N=8 specimens (N=4 in Dostar and N=4 in Placebo) that were classified into the dMMR/MSI-H subgroup but also had TP53 mutations.

For the 400 patients with mutational data available in RUBY part 1, 5 of 400 were POLE-mut (1.25%), 91 of 400 were dMMR or MSI-H (22.75%), 88 of 400 were TP53-mut (22%) and 216 of 400 were NSMP (Table 5).

TABLE 5
Prevalence of molecular subgroups in Ruby Part 1

| RUBY | | dMMR or | | |
|---|---|---|---|---|
| part 1 | POLE-mut | MSI-H | NSMP | TP53-mut |
| Prevalence % | 1.25% | 22.75% | 54% | 22% |
| (n) | (5/400) | (91/400) | (216/400) | (88/400) |

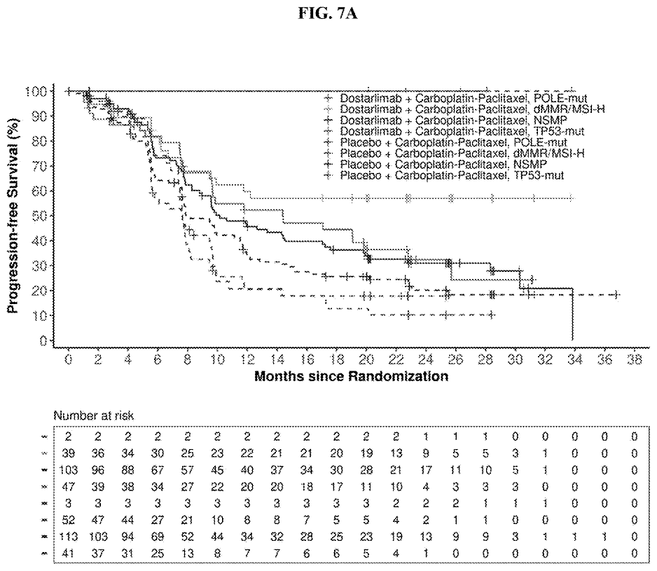

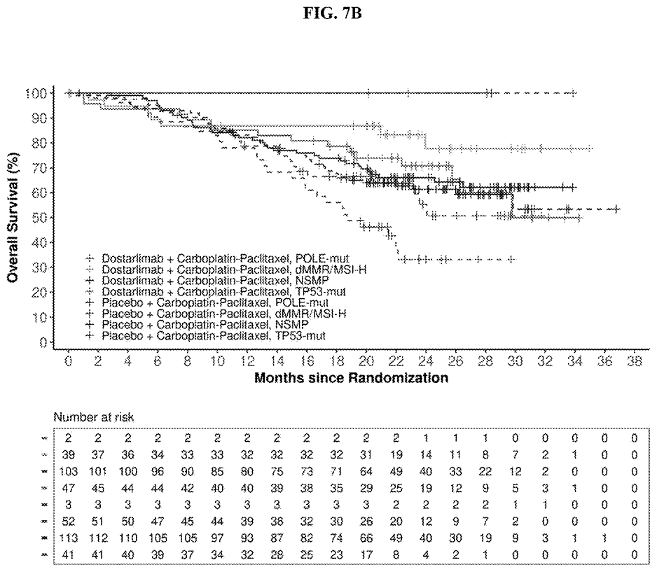

Patients with POLE-mut specimens had the best prognosis as expected, wherein none of those patients progressed either on the dostarlimab plus carboplatin-paclitaxel treatment arm (n=3) or the placebo plus carboplatin-paclitaxel treatment arm (n=2) (FIGS. 7A and 7B); as such, POLE mutation status was prognostic, and not associated with treatment benefit for the very small group of POLE-mut specimens represented in RUBY part 1.

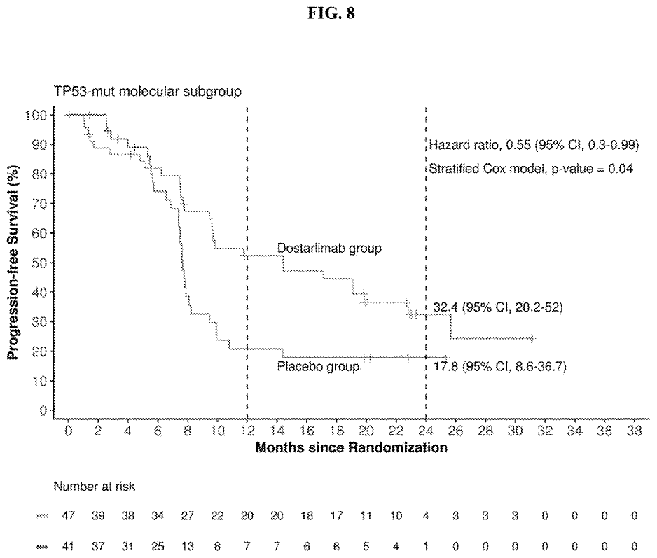

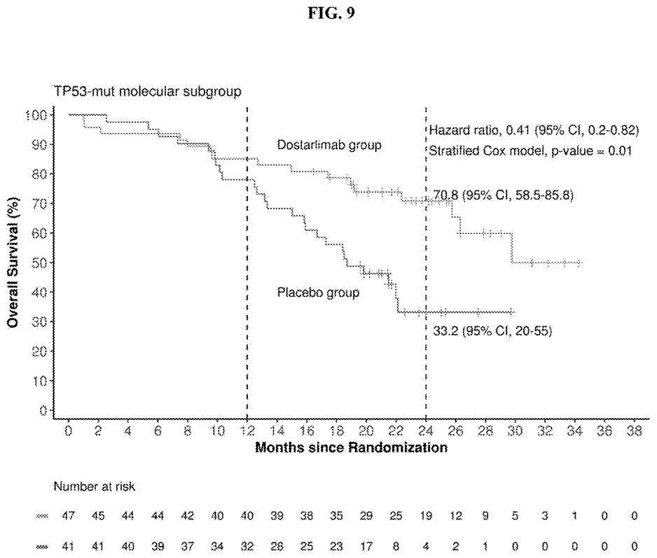

Survival analysis of TP53-mut patients in RUBY part 1 shows a statistically significant PFS (investigator assessment) benefit for patients in the dostarlimab plus carboplatin-paclitaxel treatment arm (n=47) compared with the placebo plus carboplatin-paclitaxel treatment arm (n=41), as ascertained by a stratified Cox proportional hazards model (Hazard ratio (HR)=0.55; 95% confidence interval (CI)=0.3-0.99, nominal p=0.04; FIG. 8 & Table 6). Similarly, a statistically significant OS benefit was also observed for dostarlimab plus carboplatin-paclitaxel treatment arm compared with the placebo plus carboplatin-paclitaxel treatment arm (HR=0.41; 95% CI=0.2-0.82, nominal p=0.01; FIG. 9 & Table 7).

TABLE 6
Hazard ratios across molecular subgroups comparing PFS.

| | | | Stratified | |
|---|---|---|---|---|
| | | | Cox model | |
| | | | p-value | Median PFS |
| | | | (Stratified | Dostar vs |
| | | | logrank | Placebo |
| | HR | 95% CI | test p-value) | (months) |
| POLE-mut | NE | NE | NE | NE |
| (n = 5) | | | | |
| dMMR/MSI-H | 0.31 | 0.17-0.56 | 0.0001 (0) | NA vs 7.66 |
| (n = 91) | | | | |
| TP53-mut | 0.55 | 0.3-0.99 | 0.0449 | 14.39 vs 7.66 |
| (n = 88) | | | (0.0445) | |
| NSMP | 0.77 | 0.55-1.07 | 0.1175 | 10.15 vs 7.89 |
| (n = 216) | | | (0.1173) | |

Table 6 shows the hazard ratios across molecular subgroups comparing PFS for the dostarlimab plus carboplatin-paclitaxel treatment arm versus placebo plus carboplatin-paclitaxel arm for RUBY Part 1 patients with WES data available (n=400). POLE-mut subgroup had small sample size, and therefore was not evaluated (NE).

TABLE 7
Hazard ratios across molecular subgroups comparing OS.

| | | | Stratified | |
|---|---|---|---|---|
| | | | Cox model | |
| | | | p-value | Median OS |
| | | | (Stratified | Dostar vs |
| | | | logrank | Placebo |
| | HR | 95% CI | test p-value) | (months) |
| POLE-mut | NE | NE | NE | NE |
| (n = 5) | | | | |
| dMMR/MSI-H | 0.4 | 0.17-0.95 | 0.0372 | NA vs NA |
| (n = 91) | | | (0.0313) | |
| TP53-mut | 0.41 | 0.2-0.82 | 0.0119 | 29.77 vs 18.69 |
| (n = 88) | | | (0.0099) | |
| NSMP | 0.87 | 0.56-1.36 | 0.5361 | NA vs NA |
| (n = 216) | | | (0.5358) | |

Table 7 shows the hazard ratios across molecular subgroups comparing OS for the dostarlimab plus carboplatin-paclitaxel treatment arm versus placebo plus carboplatin-paclitaxel arm for RUBY Part1 patients with WES data available (n=400). POLE-mut subgroup had small sample size, and therefore was not evaluated (NE).

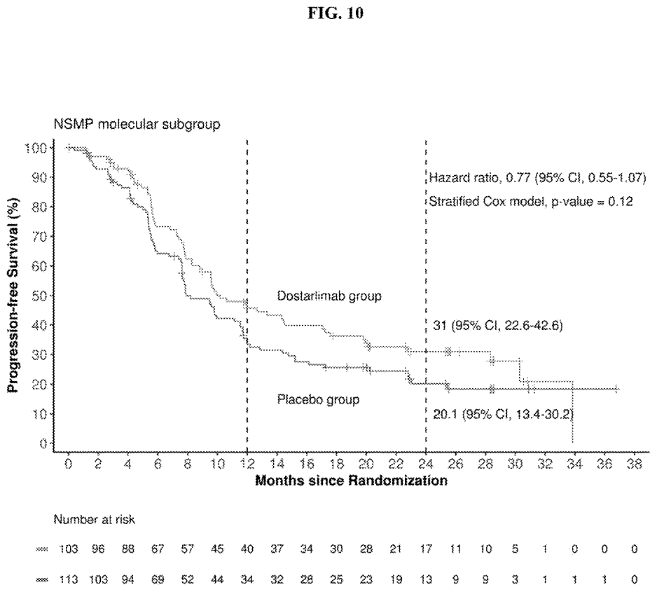

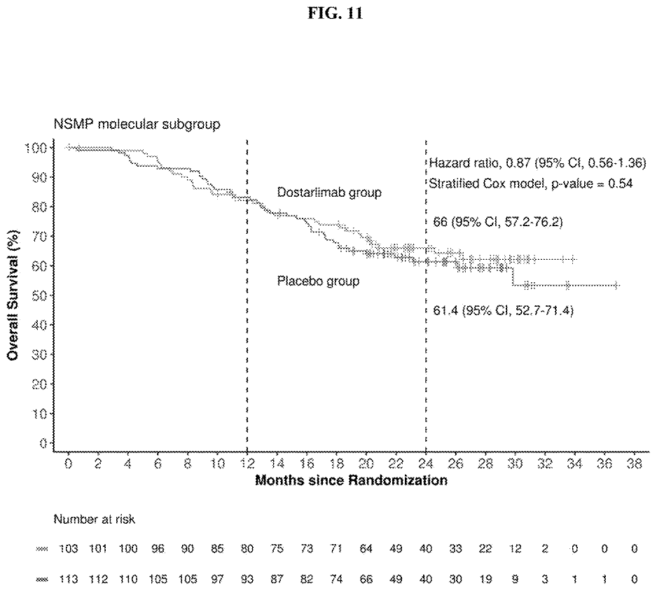

Survival analysis of NSMP patients in RUBY part 1 does not show a statistical difference in PFS (investigator assessment) benefit for patients in the dostarlimab plus carboplatin-paclitaxel treatment arm (n=103) compared with the placebo plus carboplatin-paclitaxel treatment arm (n=141), although there was a trend towards increased PFS benefit for patients treated with dostarlimab plus carboplatin-paclitaxel (HR-0.77; 95% CI=0.55-1.07, nominal p=0.12; FIG. 10 & Table 6). No OS benefit was observed for NSMP patients treated with dostarlimab plus carboplatin-paclitaxel relative to placebo plus carboplatin-paclitaxel (HR=0.87; 95% CI=0.56-1.36, nominal p=0.54; FIG. 11 & Table 7).

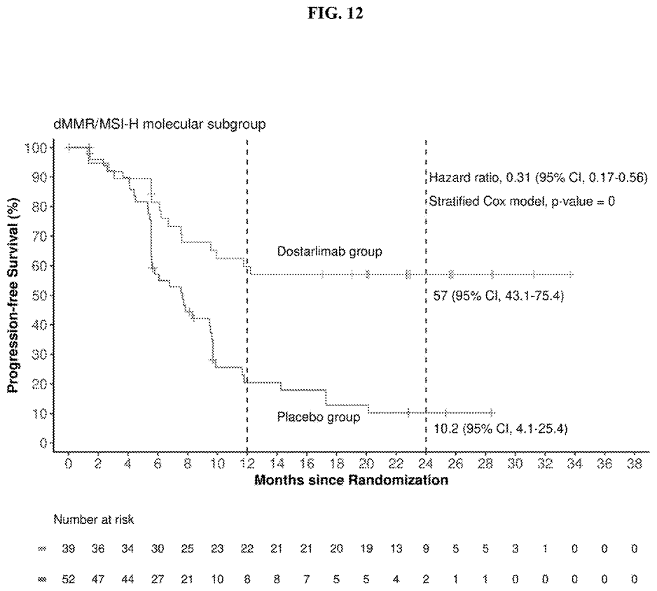

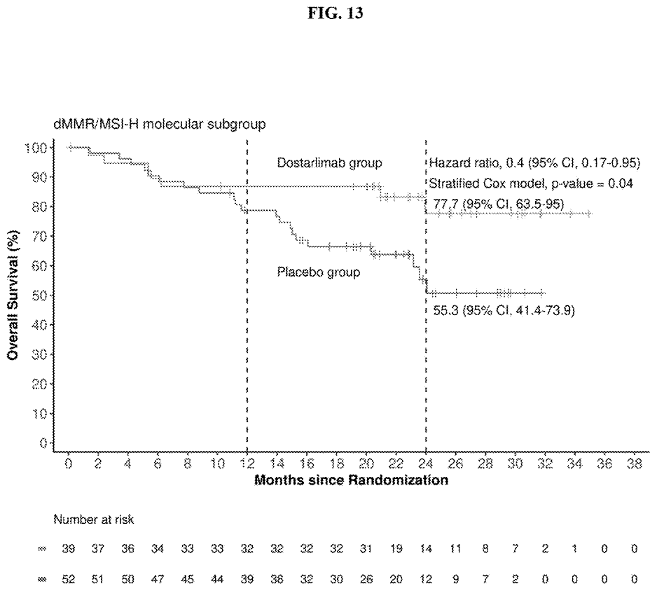

PFS (investigator assessment) improvement in the dMMR/MSI-H patient subgroup in RUBY part 1 was highly significant for the dostarlimab plus carboplatin-paclitaxel treatment arm (n=39) compared with the placebo plus carboplatin-paclitaxel treatment arm (n=52), as ascertained by a stratified Cox proportional hazards model (HR=0.31; 95% CI=0.17-0.56, nominal p=0; FIG. 12 & Table 6). Similarly, a statistically significant OS benefit was also observed for dostarlimab plus carboplatin-paclitaxel treatment arm compared with the placebo plus carboplatin-paclitaxel treatment arm (HR=0.4; 95% CI=0.17-0.95, nominal p=0.04; FIG. 13 & Table 7).

### Example 2: Treatment of TP53 Mutated Endometrial Cancer with Dostarlimab in a Patient Previously Treated with Standard of Care Chemotherapy

A TP53 mutated endometrial cancer patient previously treated with standard of care chemotherapy (e.g., carboplatin-paclitaxel) is switched to receiving treatment with dostarlimab or a biosimilar thereof, optionally in combination with standard of care chemotherapy. In some cases, the patient's treatment is switched because the patient demonstrates progressive disease after undergoing treatment with standard of care chemotherapy. In some cases, the patient's treatment is switched because the patient demonstrates only stable disease after undergoing treatment with standard of care chemotherapy. After switching to treatment with dostarlimab or a biosimilar thereof, optionally in combination with standard of care chemotherapy, the patient demonstrates an improved response.

### Example 3: Treatment of TP53 Mutated Endometrial Cancer with Dostarlimab in a Patient Previously Treated with Standard of Care Chemotherapy

An endometrial cancer patient previously treated with standard of care chemotherapy (e.g., carboplatin-paclitaxel) is switched to receiving treatment with dostarlimab or a biosimilar thereof, optionally in combination with standard of care chemotherapy. In some cases, the patient's treatment is switched because the patient demonstrates progressive disease after undergoing treatment with standard of care chemotherapy. In some cases, the patient's treatment is switched because the patient demonstrates only stable disease after undergoing treatment with standard of care chemotherapy. After switching to treatment with dostarlimab or a biosimilar thereof, optionally in combination with standard of care chemotherapy, the patient demonstrates an improved response.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, the group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of treating a TP53 mutated cancer in a human patient, the method comprising: administering a therapeutically effective amount of a combination comprising 500 mg or 1000 mg dostarlimab or a biosimilar thereof, a platinum-based chemotherapy, and paclitaxel to the human patient, such as wherein the platinum-based chemotherapy is cisplatin or carboplatin.

2. The method of claim 1, wherein the platinum-based chemotherapy comprises carboplatin.

3. The method of claim 1, wherein the administering comprises a first dose of 500 mg dostarlimab or a biosimilar thereof once every 3 weeks (Q3W) for 4 to 6 cycles, followed by a second dose of 1000 mg dostarlimab or a biosimilar thereof once every 6 weeks (Q6W).

4. The method of claim 3, wherein the second dose of dostarlimab or a biosimilar thereof is Q6W for up to 3 years.

5. (canceled)

6. (canceled)

7. (canceled)

8. The method of claim 1, wherein the administering comprises: a first dose of 500 mg dostarlimab or a biosimilar thereof once every 3 weeks (Q3W) for 6 cycles, a dose of carboplatin at an area under the concentration time curve of 5 milligram/milliliters/minute (mg/mL/min) once every 3 weeks (Q3W) for 6 cycles, and a dose of 175 milligram per meter square (mg/m²) paclitaxel once every 3 weeks (Q3W) for 6 cycles, followed by a second dose of 1000 mg dostarlimab or a biosimilar thereof once every 6 weeks (Q6W).

9. (canceled)

10. (canceled)

11. The method of claim 1, wherein the patient has not previously received treatment for the cancer.

12. The method of claim 1, wherein the cancer is gynecologic cancer.

13. The method of claim 12, wherein the gynecologic cancer is selected from endometrial cancer, ovarian cancer, fallopian tube cancer, cervical cancer, vaginal cancer, vulvar cancer, uterine cancer, uterine papillary serous carcinoma, primary peritoneal cancer, adnexal tumor, gestational trophoblastic tumor, and breast cancer.

14. (canceled)

15. (canceled)

16. The method of claim 1, wherein the cancer is recurrent or advanced endometrial cancer.

17. The method of claim 1, wherein the cancer is primary advanced endometrial cancer.

18. The method of claim 1, wherein the cancer is stage III or stage IV endometrial cancer.

19. The method of claim 1, wherein the administering results in improved progression free survival (PFS) relative to administering the platinum-based chemotherapy and paclitaxel in the absence of dostarlimab or a biosimilar thereof.

20. The method of claim 1, wherein the administering results in improved overall survival (OS) relative to administering the platinum-based chemotherapy and paclitaxel in the absence of dostarlimab or a biosimilar thereof.

21. The method of claim 1, wherein the administering results in improved duration of response (DoR) relative to administering the platinum-based chemotherapy and paclitaxel in the absence of dostarlimab or a biosimilar thereof.

22-96. (canceled)

97. A method of treating a microsatellite stable (MSS) or mismatch repair proficient (MMRp), and TP53 mutated endometrial cancer in a human patient, the method comprising: administering a therapeutically effective amount of a combination comprising 500 mg or 1000 mg dostarlimab or a biosimilar thereof, and a chemotherapy regimen.

98. The method of claim 97, wherein the administering comprises a first dose of 500 mg dostarlimab or a biosimilar thereof once every 3 weeks (Q3W) for 4 to 6 cycles, followed by a second dose of 1000 mg dostarlimab or a biosimilar thereof once every 6 weeks (Q6W).

99. The method of claim 98, wherein the endometrial cancer is recurrent or advanced endometrial cancer.

100. The method claim 98, wherein the endometrial cancer is primary advanced endometrial cancer.

101. The method of claim 98, wherein the endometrial cancer is stage III or stage IV endometrial cancer.

102. A method of treating a TP53 mutated endometrial cancer in a human patient comprising administering at least one first dose of 500 mg of dostarlimab or a biosimilar thereof every three weeks (Q3W) followed by at least one second dose of 1000 mg of dostarlimab or a biosimilar thereof every six weeks (Q6W), wherein the TP53 mutated endometrial cancer is a microsatellite stable (MSS) endometrial cancer and wherein the human patient has not been previously treated with systemic therapy for the TP53 mutated endometrial cancer.

103. The method of claim 102 wherein the TP53 mutated endometrial cancer is primary advanced endometrial cancer or stage III or stage IV endometrial cancer.

104. The method of claim 102 wherein the progression free survival (PFS) is improved relative to administering a platinum-based chemotherapy in the absence of dostarlimab or a biosimilar thereof.
